SEQUENCE LISTING (69 sequences)

SEQ ID NO: 1 - DNA, 18 bp, Artificial Sequence
  vector sequence
agccatatgcagatcttc

SEQ ID NO: 2 - DNA, 21 bp, Artificial Sequence
  vector sequence
ctccgcggtggatccgaattc

SEQ ID NO: 3 - protein, 4 aa, Artificial Sequence
  vector sequence
MQIF

SEQ ID NO: 4 - protein, 4 aa, Artificial Sequence
  vector sequence
LRGG

SEQ ID NO: 5 - DNA, 18 bp, Artificial Sequence
  vector sequence
caccatcagatcttcgtc

SEQ ID NO: 6 - protein, 4 aa, Artificial Sequence
  vector sequence
QIFV

SEQ ID NO: 7 - DNA, 18 bp, Artificial Sequence
  vector sequence
caccatcagctcttcatc

SEQ ID NO: 8 - protein, 4 aa, Artificial Sequence
  vector sequence
QLFI

SEQ ID NO: 9 - protein, 76 aa, Homo Sapiens [sequence omitted]

SEQ ID NO: 10 - protein, 76 aa, Artificial Sequence
  mutant ubiquitin protein
MQLFIKTLTGKTVTLEVEPSDTIENLKAKLQDKEGIPPDQQRLIFAGKQLEDGRTLSDYNIQKESTLHIVLRLRGG

SEQ ID NO: 11 - protein, 76 aa, Artificial Sequence
  mutant ubiquitin protein
MQIFVKTLTGKTITIEIEPSDTIENVKAKLQDKEGIPPDQQRIIFAGKQIEDGRTISDYNIQKESTLHLVLRLRGG

SEQ ID NO: 12 - protein, 4 aa, Artificial Sequence
  vector sequence
MGSS

SEQ ID NO: 13 - protein, 6 aa, Artificial Sequence
  vector sequence
HHHHHH

SEQ ID NO: 14 - protein, 8 aa, Artificial Sequence
  vector sequence
HHHHHHHH

SEQ ID NO: 15 - protein, 66 aa, Artificial Sequence
  primer sequence
CTTGATAGCCATATGGGCTCTTCCCACCATCACCATCACCATCAGATCTTCGTCAAGACGTTAACC

SEQ ID NO: 16 - DNA, 55 bp, Artificial Sequence
  primer sequence
gcgatagaattcggatccaccgcggagacgtaagacaagatgtaaggtcgactcc

SEQ ID NO: 17 - DNA, 72 bp, Artificial Sequence
  primer sequence
cttgatagccatatgggctcttcccaccatcaccatcaccatcaccatcagctcttcatcaagacgttaacc

SEQ ID NO: 18 - DNA, 55 bp, Artificial Sequence
  primer sequence
gcgatagaattcggatccaccgcggagacgtaagacaatatgtaaggtcgactcc

SEQ ID NO: 19 - DNA, 480 bp, Artificial Sequence
  vector sequence
atgggctcttcccaccatcaccatcaccatcaccatcagctcttcgtcaagacgttaaccggtaaaaccataactctagaacttgaaccatccgataccgtcgaaaacgttaaggctaaaattcaagacaaggaaggtattccacctgatcaacaacgtttgatctttgccggtaagcagctcgaagacggtcgtacgctgtctgattacaacattcagaaggagtcgaccttacatcttgtcttacgtctccgcggtggagcaccaatgggctctgaccctcccaccgcctgctgcttttcttacaccgcgaggaaacttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaattgatgaaagctt

SEQ ID NO: 20 - protein, 156 aa, Artificial Sequence
  vector sequence
MGSSHHHHHHHHQLFVKTLTGKTITLELEPSDTVENVKAKIQDKEGIPPDQQRLIFAGKQLEDGRTLSDYNIQKESTLHLVLRLRGGAPMGSDPPTACCFSYTARKLPRNFVVDYYETSSLCSQPAVVFQTKRSKQVCADPSESWVQEYVYDLELN

SEQ ID NO: 21 - DNA, 501 bp, Artificial Sequence
  vector sequence
atgggcagcagccatcatcatcatcatcacagcagcggcctggtgccgcgcggcagccatatgcagatctttgtgaagaccctcactggcaaaaccatcacccttgaggtcgagcccagtgacaccattgagaatgtcaaagccaaaattcaagacaaggagggtatcccacctgaccagcagcgtctgatatttgccggcaaacagctggaggatggccgcactctctcagactacaacatccagaaagagtccaccctgcacctggtgttgcgcctccgcggtggagcaccaatgggctctgaccctcccaccgcctgctgcttttcttacaccgcgaggaaacttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaattgatga

SEQ ID NO: 22 - protein, 165 aa, Artificial Sequence
  vector sequence
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGAPMGSDPPTACCFSYTARKLPRNFVVDYYETSSLCSQPAVVFQTKRSKQVCADPSESWVQEYVYDLELN

SEQ ID NO: 23 - protein, 19 aa, Artificial Sequence
  vector sequence
KLGSGLNDIFEAQKIEWHE

SEQ ID NO: 24 - protein, 96 aa, Artificial Sequence
  vector sequence
MGSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGG

SEQ ID NO: 25 - protein, 87 aa, Artificial Sequence
  vector sequence
MGSSHHHHHHHHQLFVKTLTGKTITLELEPSDTVENVKAKIQDKEGIPPDQQRLIFAGKQLEDGRTLSDYNIQKESTLHLVLRLRGG

SEQ ID NO: 26 - protein, 75 aa, Artificial Sequence
  mutant ubiquitin sequence
QLFVKTLTGKTITLELEPSDTVENVKAKIQDKEGIPPDQQRLIFAGKQLEDGRTLSDYNIQKESTLHLVLRLRGG

SEQ ID NO: 27 - protein, 99 aa, Homo Sapiens
  PEPTIDE (1)...(95): CCL2
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 28 - protein, 92 aa, Homo Sapiens
  PEPTIDE (1)...(90): CCL3
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 29 - protein, 99 aa, Homo Sapiens
  PEPTIDE (1)...(95): CCL7
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 30 - protein, 98 aa, Homo Sapiens
  PEPTIDE (1)...(95): CCL13
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 31 - protein, 93 aa, Homo Sapiens
  PEPTIDE (1)...(90): CCL14
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 32 - protein, 112 aa, Homo Sapiens
  PEPTIDE (1)...(110): CCL27
MKGPPTFCSLLLLSLLLSPDPTAAFLLPPSTACCTQLYRKPLSDKLLRKVIQVELQEADGDCHLQAFVLHLAQRSICIHPQNPSLSQWFEHQERKLHGTLPKLNFGMLRKMG

SEQ ID NO: 33 - protein, 127 aa, Homo Sapiens
  PEPTIDE (1)...(125): CCL28
MQQRGLAIVALAVCAALHASEAILPIASSCCTEVSHHISRRLLERVNMCRIQRADGDCDLAAVILHVKRRRICVSPHNHTVKQWMKVQAAKKNGKGNVCHRKKHHGKRNSNRAHQGKHETYGHKTPY

SEQ ID NO: 34 - protein, 99 aa, Homo Sapiens
  PEPTIDE (1)...(95): CXCL8
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 35 - protein, 125 aa, Homo Sapiens
  PEPTIDE (1)...(125): CXCL9
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 36 - protein, 98 aa, Homo Sapiens
  PEPTIDE (1)...(95): CXCL10
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 37 - protein, 94 aa, Homo Sapiens
  PEPTIDE (1)...(90): CXCL11
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 38 - protein, 111 aa, Homo Sapiens
  PEPTIDE (1)...(110): CCL14
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 39 - protein, 73 aa, Homo Sapiens
  PEPTIDE (0)...(0)
ASVATELRCQCLQTLQGIHPKNIQSVNVKSPGPHCAQTEVIATLKNGRKACLNPASPIVKKIIEKMLNSDKSN

SEQ ID NO: 40 - protein, 73 aa, Homo Sapiens
  PEPTIDE (0)...(0)
APLATELRCQCLQTLQGIHLKNIQSVKVKSPGPHCAQTEVIATLKNGQKACLNPASPMVKKIIEKMLKNGKSN

SEQ ID NO: 41 - protein, 73 aa, Homo Sapiens
  PEPTIDE (0)...(0)
ASVVTELRCQCLQTLQGIHLKNIQSVNVRSPGPHCAQTEVIATLKNGKKACLNPASPMVQKIIEKILNKGSTN

SEQ ID NO: 42 - protein, 70 aa, Homo Sapiens
  PEPTIDE (0)...(0)
EAEEDGDLQCLCVKTTSQVRPRHITSLEVIKAGPHCPTAQLIATLKNGRKICLDLQAPLYKKIIKKLLES

SEQ ID NO: 43 - protein, 78 aa, Homo Sapiens
  PEPTIDE (0)...(0)
AGPAAAVLRELRCVCLQTTQGVHPKMISNLQVFAIGPQCSKVEVVASLKNGKEICLDPEAPFLKKVIQKILDGGNKEN

SEQ ID NO: 44 - protein, 77 aa, Homo Sapiens
  PEPTIDE (0)...(0)
GPVSAVLTELRCTCLRVTLRVNPKTIGKLQVFPAGPQCSKVEVVASLKNGKQVCLDPEAPFLKKVIQKILDSGNKKN

SEQ ID NO: 45 - protein, 73 aa, Homo Sapiens; identical to SEQ ID NO: 41
  PEPTIDE (0)...(0)

SEQ ID NO: 46 - protein, 68 aa, Homo Sapiens
  PEPTIDE (0)...(0)
KPVSLSYRCPCRFFESHVARANVKHLKILNTPNCALQIVARLKNNNRQVCIDPKLKWIQEYLEKALNK

SEQ ID NO: 47 - protein, 109 aa, Homo Sapiens
  PEPTIDE (0)...(0)
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 48 - protein, 77 aa, Homo Sapiens
  PEPTIDE (0)...(0)
SKCKCSRKGPKIRYSDVKKLEMKPKYPHCEEKMVIITTKSVSRYRGQEHCLHPKLQSTKRFIKWYNAWNEKRRVYEE

SEQ ID NO: 49 - protein, 225 aa, Homo Sapiens
  PEPTIDE (0)...(0)
NEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 50 - protein, 92 aa, Homo Sapiens
  PEPTIDE (0)...(0)
GSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 51 - protein, 93 aa, Homo Sapiens
  PEPTIDE (0)...(0)
VGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 52 - protein, 76 aa, Homo Sapiens
  PEPTIDE (0)...(0)
QAHGVTKCNITCSKMTSKIPVALLIHYQQNQASCGKRAIILETRQHRLFCADPKEQWVKDAMQHLDRQAAALTRNG

SEQ ID NO: 53 - protein, 73 aa, Homo Sapiens
  PEPTIDE (0)...(0)
KSMQVPFSRCCFSFAEQEIPLRAILCYRNTSSICSNEGLIFKLKRGKEACALDTVGWVQRHRKMLRHCPSKRK

SEQ ID NO: 54 - protein, 69 aa, Homo Sapiens
  PEPTIDE (0)...(0)
APMGSDPPTACCFSYTARKLPRNFVVDYYETSSLCSQPAVVFQTKRSKQVCADPSESWVQEYVYDLELN

SEQ ID NO: 55 - protein, 69 aa, Homo Sapiens
  PEPTIDE (0)...(0)
MSPYSSDTTPCCFAYIARPLPRAHIKEYFYTSGKCSNPAVVFVTRKNRQVCANPEKKWVREYINSLEMS

SEQ ID NO: 56 - protein, 77 aa, Homo Sapiens
  PEPTIDE (0)...(0)
MQPDSVSIPITCCFNVINRKIPIQRLESYTRITNIQCPKEAVIFKTKRGKEVCADPKERWVRDSMKHLDQIFQNLKP

SEQ ID NO: 57 - protein, 73 aa, Homo Sapiens; identical to SEQ ID NO: 41
  PEPTIDE (0)...(0)

SEQ ID NO: 58 - protein, 92 aa, Homo Sapiens
  PEPTIDE (0)...(0)
QFINDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 59 - protein, 97 aa, Homo Sapiens
  PEPTIDE (0)...(0)
QPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 60 - protein, 71 aa, Homo Sapiens
  PEPTIDE (0)...(0)
ARGTNVGRECCLEYFKGAIPLRKLKTWYQTSEDCSRDAIVFVTVQGRAICSDPNNKRVKNAVKYLQSLERS

SEQ ID NO: 61 - protein, 69 aa, Homo Sapiens
  PEPTIDE (0)...(0)
AQVGTNKELCCLVYTSWQIPQKFIVDYSETSPQCPKPGVILLTKRGRQICADPNKKWVQKYISDLKLNA

SEQ ID NO: 62 - protein, 77 aa, Homo Sapiens
  PEPTIDE (0)...(0)
GTNDAEDCCLSVTQKPIPGYIVRNFHYLLIKDGCRVPAVVFTTLRGRQLCAPPDQPWVERIIQRLQRTSAKMKRRSS

SEQ ID NO: 63 - protein, 70 aa, Homo Sapiens
  PEPTIDE (0)...(0)
ASNFDCCLGYTDRILHPKFIVGFTRQLANEGCDINAIIFHTKKKLSVCANPKQTWVKYIVRLLSKKVKNM

SEQ ID NO: 64 - protein, 110 aa, Homo Sapiens
  PEPTIDE (0)...(0)
SDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 65 - protein, 69 aa, Homo Sapiens
  PEPTIDE (0)...(0)
GPYGANMEDSVCCRDYVRYRLPLRVVKHFYWTSDSCPRPGVVLLTFRDKEICADPRVPWVKMILNKLSQ

SEQ ID NO: 66 - protein, 99 aa, Homo Sapiens
  PEPTIDE (0)...(0)
RVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 67 - protein, 78 aa, Homo Sapiens
  PEPTIDE (0)...(0)
VVIPSPCCMFFVSKRIPENRVVSYQLSSRSTCLKAGVIFTTKKGQQFCGDPKQEWVQRYMKNLDAKQKKASPRARAVA

SEQ ID NO: 68 - protein, 128 aa, Homo Sapiens
  PEPTIDE (0)...(0)
TQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 69 - protein, 71 aa, Homo Sapiens
  PEPTIDE (0)...(0)
TRGSDISKTCCFQYSHKPLPWTWVRSYEFTSNSCSQRAVIFTTKRGKKVCTHPRKKWVQKYISLLKTPKQL

(19) United States
(12) Patent Application Publication
(10) Pub. No.: US 2014/0045211 A1
(43) Pub. Date: Feb. 13, 2014

(54) HIGH LEVEL PRODUCTION OF RECOMBINANT PROTEINS

(71) Applicants: Tracy M. Handel, San Diego, CA (US); Andrew Douglas, San Diego, CA (US)

(72) Inventors: Tracy M. Handel, San Diego, CA (US); Andrew Douglas, San Diego, CA (US)

(73) Assignee: The Regents of the University of California, Oakland, CA (US)

(21) Appl. No.: 14/006,587

(22) PCT Filed: Mar. 22, 2012
(86) PCT No.: PCT/US2012/030208

§ 371 Date: Oct. 30, 2013

Related U.S. Application Data
(60) Provisional application No. 61/467,270, filed on Mar. 24, 2011.

(51) Int. Cl. C07K 14/52; C07K 14/00

(52) U.S. Cl. CPC C07K 14/521; C07K 14/00
USPC 435/68.1; 435/69.7; 435/252.33; 530/350; 536/23.4

(57) ABSTRACT

The present technology relates to the fields of biochemistry, molecular biology and medicine. In particular, the present technology relates to methods and compositions for increased expression of recombinant proteins.

### CROSS-REFERENCE TO RELATED APPLICATIONS

This application claims the benefit of U.S. Provisional Application No. 61/467,270 entitled “HIGH LEVEL PRODUCTION OF RECOMBINANT PROTEINS” filed Mar. 24, 2011, the disclosure of which is hereby expressly incorporated by reference in its entirety.

### STATEMENT REGARDING FEDERALLY SPONSORED R&D

This invention was made with government support under Grant No. RO1-AI37113, RO1-GM081763, and U01-GM094612 awarded by NIH. The government has certain rights in the invention.

### REFERENCE TO SEQUENCE LISTING

The present application is being filed along with a Sequence Listing in electronic format. The Sequence Listing is provided as a file entitled UCSD028_{—}002PR.TXT, created Mar. 21, 2012, which is approximately 48 KB in size. The information in the electronic format of the Sequence Listing is incorporated herein by reference in its entirety.

### FIELD OF THE INVENTION

The present technology relates to the fields of biochemistry, molecular biology and medicine. In particular, the present technology relates to methods and compositions for increased expression of recombinant proteins.

### BACKGROUND

Recombinant proteins have an increasingly diverse number of uses in industrial processes, therapeutic methods, and basic research. In many such uses, high quantities of a recombinant protein may be required. The isolation of high levels of a recombinant protein requires an efficient expression system. Unfortunately, high-level expression of biologically active recombinant proteins, especially recombinant eukaryotic proteins, is often difficult to achieve.

Several systems have emerged that include fusing a first gene encoding a polypeptide of interest downstream of a second gene encoding a second polypeptide to produce a single recombinant fusion protein (See e.g., Uhlen, M. et al., 1990. Gene fusions for purpose of expression: An introduction. Methods Enzymol. 185: 129-143). Generally, such strategies can provide increased protein yields and may allow simple purification methods due to the affinity of certain fusion partners for a particular ligand (Baker, R. T. 1996. Protein expression using ubiquitin fusion and cleavage. Curr. Opin. Biotechnol. 7: 541-546). A drawback of such strategies is the covalent linkage of the two proteins, where the presence of the fusion partner may prevent or interfere with subsequent use of the polypeptide of interest. To overcome this problem a protease recognition site can be inserted between the two fused polypeptides; however, this involves altering the N terminus of the desired product, resulting in the expression of an unauthentic protein (Butt, T. R., et al., 1989. Ubiquitin fusion augments the yield of cloned gene products in Escherichia coli. Proc. Natl. Acad. Sci. 86: 2540-2544). Furthermore, cleavage of the fusion protein is rarely complete, causing a reduction in protein yield, and it may also occur nonspecifically within the fused protein (Baker, R. T. 1996. Protein expression using ubiquitin fusion and cleavage. Curr. Opin. Biotechnol. 7: 541-546).

### SUMMARY

Some embodiments of the methods and compositions provided herein include a method for producing a recombinant protein comprising producing a fusion protein in a cell, wherein said fusion protein comprises a protein of interest and a modified protein moiety, wherein said modified protein moiety decreases the solubility of said fusion protein in said cell compared to the solubility of said protein of interest in said cell.

Some embodiments also include cleaving said protein of interest from said modified protein moiety. In some embodiments, said cleaving comprises utilizing a protease. In some embodiments, said protease is ubiquitinase. In some embodiments, said ubiquitinase comprises Usp2-cc.

Some embodiments also include providing conditions for said protein of interest to refold into an active form, wherein said protein of interest is cleaved from said modified protein moiety.

Some embodiments also include purifying said fusion protein or said protein of interest.

In some embodiments, said modified protein moiety comprises a modified ubiquitin moiety.

In some embodiments, said modified ubiquitin moiety comprises an increased frequency or number of hydrophobic amino acid residues compared to a wild type ubiquitin moiety.

In some embodiments, said modified ubiquitin moiety comprises a decreased frequency or number of hydrophobic amino acid residues compared to a wild type ubiquitin moiety.

In some embodiments, said modified ubiquitin moiety comprises a ubiquitin sequence with one or more mutations at positions selected from the group consisting of I3, V5, I13, L15, V17, I23, V26, I30, L43, L50, L56, and L69. In some embodiments, said modified ubiquitin moiety comprises a ubiquitin sequence with one or more mutations selected from the group consisting of I3L, I13V, V5I, L15I, V17L, I23V, V26L, I30L, L43I, L50I, L56I, and L69I. In some embodiments, said modified ubiquitin moiety comprises a ubiquitin sequence with one or more mutations selected from the group consisting of I3L, V17L, and I23V. In some embodiments, said modified ubiquitin moiety comprises a ubiquitin sequence with one or more mutations selected from the group consisting of I3L, V51, I13V, V26L, I30L, and L69I. In some embodiments, said modified ubiquitin moiety comprises a ubiquitin sequence with one or more mutations selected from the group consisting of L15I, V17I, I30L, L43I, L50I, and L56I.

In some embodiments, said modified ubiquitin moiety comprises a sequence selected from the groups consisting of SEQ ID NO:10, SEQ ID NO:11, and SEQ ID NO:26.

In some embodiments, said protein of interest is a soluble protein.

In some embodiments, said protein of interest is selected from the group consisting of secreted proteins, cytoplasmic proteins, nuclear proteins, and membrane proteins.

In some embodiments, said protein of interest is a chemokine or a cytokine. In some embodiments, the protein of interest comprises a polypeptide selected from the group consisting of SEQ ID NO.s::27-69.

In some embodiments, said protein of interest is toxic to said cell, wherein said protein of interest is in a soluble form.

In some embodiments, said protein of interest is degraded by said cell, wherein said protein of interest is in a soluble form.

In some embodiments, said protein of interest is encoded by a nucleic acid sequence codon-optimized for expression in said cell.

In some embodiments, said cell is prokaryotic.

Some embodiments of the methods and compositions provided herein include a recombinant protein produced by any of the methods provided herein.

Some embodiments of the methods and compositions provided herein include a fusion protein comprising a protein of interest and a modified protein moiety, wherein said modified protein moiety decreases the solubility of said fusion protein in a cell compared to the solubility of said protein of interest in said cell.

In some embodiments, said modified protein moiety comprises a modified ubiquitin moiety.

In some embodiments, said modified ubiquitin moiety comprises an increased frequency of hydrophobic amino acid residues compared to a wild type ubiquitin moiety.

In some embodiments, said modified ubiquitin moiety comprises a decreased frequency or number of hydrophobic amino acid residues compared to a wild type ubiquitin moiety.

In some embodiments, modified ubiquitin moiety comprises a ubiquitin sequence with one or more mutations at positions selected from the group consisting of I3, V5, I13, L15, V17, I23, V26, I30, L43, L50, L56, and L69. In some embodiments, said modified ubiquitin moiety comprises a ubiquitin sequence with one or more mutations selected from the group consisting of I3L, I13V, V5I, L15I, V17L, I23V, V26L, I30L, L43I, L50I, L56I, and L69I. In some embodiments, said modified ubiquitin moiety comprises a ubiquitin sequence comprising one or more mutations selected from the group consisting of I3L, V17L, and 123V. In some embodiments, said modified ubiquitin moiety comprises a ubiquitin sequence with one or more mutations selected from the group consisting of I3L, V5I, I13V, V26L, I30L, and L69I. In some embodiments, said modified ubiquitin moiety comprises a ubiquitin sequence with one or more mutations selected from the group consisting of L15I, V17I, I30L, L43I, L50I, and L56I.

In some embodiments, said modified ubiquitin moiety comprises a sequence selected from the groups consisting of SEQ ID NO:10, SEQ ID NO:11, and SEQ ID NO:26.

In some embodiments, said protein of interest is a soluble protein.

In some embodiments, said protein of interest is selected from the group consisting of secreted proteins, cytoplasmic proteins, nuclear proteins, and membrane proteins.

In some embodiments, said protein of interest is a chemokine or a cytokine In some embodiments, the protein of interest comprises a polypeptide selected from the group consisting of SEQ ID NO.s: 27-69.

In some embodiments, said protein of interest is toxic to a cell, wherein said cell expresses said fusion protein.

In some embodiments, said protein of interest is degraded by a cell, wherein said cell expresses said fusion protein.

In some embodiments, said protein of interest is encoded by a nucleic acid sequence codon-optimized for expression in said cell.

In some embodiments, said cell is prokaryotic.

Some embodiments of the methods and compositions provided herein include a nucleic acid encoding any of the fusion proteins provided herein.

Some embodiments of the methods and compositions provided herein include a cell comprising any of the nucleic acids provided herein.

Some embodiments of the methods and compositions provided herein include a method of obtaining a recombinant protein comprising: providing a nucleic acid comprising a sequence encoding a modified ubiquitin moiety and a protein of interest, wherein the sequence encoding the modified ubiquitin moiety decreases the solubility of the protein of interest; and expressing the nucleic acid in a cell.

Some embodiments also include cleaving the modified ubiquitin moiety from the expression product of the nucleic acid. In some embodiments, the cleaving comprises contacting an ubiquitinase with said expression product. In some embodiments, the ubiquitinase comprises Usp2-cc.

Some embodiments also include providing conditions for said expression product of said nucleic acid to refold into an active form.

In some embodiments, said modified ubiquitin moiety comprises an increased frequency or number of hydrophobic amino acid residues compared to a wild type ubiquitin moiety.

In some embodiments, said modified ubiquitin moiety comprises a decreased frequency or number of hydrophobic amino acid residues compared to a wild type ubiquitin moiety.

In some embodiments, said modified ubiquitin moiety comprises a ubiquitin sequence with one or more mutations at positions selected from the group consisting of I3, V5, I13, L15, V17, I23, V26, I30, L43, L50, L56, and L69. In some embodiments, said modified ubiquitin moiety comprises a ubiquitin sequence with one or more mutations selected from the group consisting of I3L, I13V, V5I, L15I, V17L, I23V, V26L, I30L, L43I, L50I, L56I, and L69I. In some embodiments, said modified ubiquitin moiety comprises an ubiquitin sequence comprising one or more mutations selected from the group consisting of I3L, V17L, and 123V. In some embodiments, said modified ubiquitin moiety comprises a ubiquitin sequence with one or more mutations selected from the group consisting of I3L, V5I, I13V, V26L, I30L, and L69I. In some embodiments, said modified ubiquitin moiety comprises a ubiquitin sequence with one or more mutations selected from the group consisting of L15I, V17I, I30L, L43I, L50I, and L56I.

In some embodiments, said modified ubiquitin moiety comprises a sequence selected from the groups consisting of SEQ ID NO:10, SEQ ID NO:11, and SEQ ID NO:26.

In some embodiments, said protein of interest is a soluble protein.

In some embodiments, said protein of interest is selected from the group consisting of secreted proteins, cytoplasmic proteins, nuclear proteins, and membrane proteins.

In some embodiments, said protein of interest is a chemokine or a cytokine. In some embodiments, the protein of interest comprises a polypeptide selected from the group consisting of SEQ ID NO.s: 27-69.

In some embodiments, said protein of interest is toxic to said cell, wherein said protein of interest is in a soluble form.

In some embodiments, said protein of interest is degraded by said cell, wherein said protein of interest is in a soluble form.

In some embodiments, the nucleic acid encoding the protein of interest encodes a sequence codon-optimized for expression in said cell.

In some embodiments, said cell is prokaryotic.

### BRIEF DESCRIPTION OF THE DRAWINGS

FIG. 1 shows a schematic for vectors for recombinant fusion proteins containing ubiquitin sequences. Top: pHUE vector including 5′ or 3′ of ubiquitin coding sequences, SEQ ID NO:01 and SEQ ID NO:02, and portions of the encoded amino acid sequences, SEQ ID NO:03 and SEQ ID NO:04, respectively. Middle: pCEV_{opt/R6} vector including 5′ or 3′ of ubiquitin coding sequences, SEQ ID NO:05 and SEQ ID NO:02, and portions of the encoded amino acid sequences, SEQ ID NO:06 and SEQ ID NO:04, respectively. Bottom: pCEV_{2D6} vector including 5′ or 3′ of ubiquitin coding sequences, SEQ ID NO:07 and SEQ ID NO:02, and portions of the encoded amino acid sequences, SEQ ID NO:8 and SEQ ID NO:04, respectively. The T7 promoter of each vector is shown as a filled arrow, an ubiquitinase cleavage site after second glycine in the encoded polypeptide is indicated with a grey arrow.

FIG. 2 shows sequence alignment of wildtype ubiquitin (SEQ ID NO:09), the 2D6 mutant ubiquitin (SEQ ID NO:10), and the R6 mutant ubiquitin (SEQ ID NO:11).

FIG. 3A, FIG. 3B, FIG. 3C, FIG. 3D, FIG. 3E, and FIG. 3F show photographs of SDS-PAGE gels for protein expression of ubiquitin-chemokine fusion proteins from various vectors. Expression fractions were taken at 0, 1, 2, and 4 hrs post induction. The full length ubiquitin-chemokine is indicated with arrows. FIG. 3A shows expression from pCEV-05. FIG. 3B shows expression from pCEV-01, pCEV-06, pCEV-11, and pCEV-16. FIG. 3C shows expression from pCEV-02, pCEV-07, pCEV-12, and pCEV-17FIG. 3D shows expression from pCEV-03, pCEV-08, pCEV-13, and pCEV-18. FIG. 3E shows expression from pCEV-04, pCEV-09, pCEV-14, and pCEV-19. FIG. 3F shows expression from pCEV-05, pCEV-10, pCEV-15, and pCEV-20.

FIG. 4 shows photographs of SDS-PAGE gels for protein expression of ubiquitin-chemokine fusion proteins from (A) pCEV-04 encoding Ub_{WT}-CCL7, and (B) pCEV-09 encoding Ubₒₚₜ-CCL7. Expression fractions were taken at 0, 1, 2, and 4 hrs post induction.

FIG. 5 shows photographs of SDS-PAGE gels for protein expression of ubiquitin-chemokine fusion proteins from (A) pCEV-14 encoding Ub_{2D6}-CCL7, and (B) pCEV-19 encoding Ub_{R6}-CCL7. Expression fractions were taken at 0, 1, 2, and 4 hrs post induction.

FIG. 6 shows a schematic of an embodiment of the present technology, and the pHUE system (the Baker system) (Catanzariti, A.-M., et al., An efficient system for high-level expression and easy purification of authentic recombinant proteins. Protein Sci, 2004. 13(5): p. 1331-1339). The modified system includes the use of a core-redesigned fusion protein partner (for example, an ubiquitin partner) that promotes inclusion body expression of a protein of interest fused thereto. Inclusion body expression and codon optimization of the fusion protein help to increase the overall yield of fusion protein. Inclusion body expression of the protein of interest can avoid problems involved with toxicity or protease susceptibility. The protein of interest can be cleaved from the fusion by ubiquitinase to generate protein of interest with precisely defined termini.

FIG. 7 shows photographs of two SDS-PAGE gels for expression of soluble and insoluble protein fractions over time. Redesigned ubiquitin can be used to shift protein from the soluble to the insoluble fraction. On the left is the pHUE system (R. Baker System) where most of the protein (the bands highlighted by **) is in the soluble fractions at Time=1.5 and 3 hrs. On the right is the redesigned ubiquitin system where most of the protein is in the insoluble fraction (the bands highlighted by **). Protein produced with the redesigned ubiquitin is insoluble.

FIG. 8 depicts expression of multiple chemokines as ubiquitin fusions. Test expressions of ubiquitin-CCL7 fusions in BL21(DE3)-pLysS cells. Supernatant (S) and pellet (P) fractions for samples taken before induction (T₀) and after 4 hrs (T₄) were run on 15% SDS-PAGE gels and stained with Coomassie Blue.

FIG. 9 depicts purification and functional characterization of CCL7. (A) SDS-PAGE analysis of IMAC purification of ubiquitin-CCL7 fusion. Samples were separated on a 15% SDS-PAGE gel and stained with Coomassie Blue. The ubiquitin-CCL7 fusion is indicated by an arrow. (B) SDS-PAGE analysis of an Usp2-cc cleavage time course. The ubiquitin-CCL7 fusion was cleaved using Usp2-cc at a 100:1 molar ratio. Samples were taken after 0, 1, 2, 4 and 20 hrs, separated using a 15% SDS-PAGE gel and stained with Coomassie Blue. Cleaved CCL7, indicated by an arrow, runs slightly more slowly than cleaved ubiquitin. (C) Comparison of calcium flux induced by the addition of purified CCL7 and commercial CCL7. Increasing concentrations of CCL7, either purified in house (solid, black circles), or purchased from R & D Systems (open triangles) were added to HEK293 cells stably expressing CCR1. Maximum increases in fluorescence of a Ca2+ sensitive dye are plotted as a function of CCL7 concentration.

FIG. 10 depicts destabilized variants of ubiquitin resulting in insoluble expression of CCL7-fusions. CCL7 was expressed as a fusion with either wildtype ubiquitin (A), codon-optimized ubiquitin (B) or increasingly destabilizing ubiquitin mutants, 3D3 (C), 2D6 (D) or R6 (E). Supernatant (S) and pellet (P) fractions for samples taken before induction (Tₒ) and after 4 hrs (T₄) were run on 15% SDS-PAGE gels and stained with Coomassie Blue. Ubiquitin-CCL7 fusions are indicated by arrows.

FIG. 11 depicts a comparison of cleavage efficiencies of destabilized ubiquitin fusions. SDS-PAGE analysis of Usp2-cc cleavage time courses of ubiquitin-CCL7-variants. CCL7 fused to wildtype ubiquitin (CCL7-WT) or a destabilizing ubiquitin variant (CCL7-2D6 or CCL7-3D3) was cleaved using Usp2-cc as described herein. Samples of the cleavage reaction were taken prior to addition of Usp2-cc (T₀), or 1 hr (T₁)/12 hrs (T₁₂) after the addition of Usp2-cc. Proteins were separated on a 15% SDS-PAGE gel and stained with Coomassie Blue. Bands corresponding to Usp2-cc, ubiquitin-CCL7 fusion (Ub-CCL7), ubiquitin (Ub) and CCL7 are indicated by arrows.

FIG. 12 depicts a schematic and purification scheme for fusion polypeptides such as chemokines (A) Schematic of chemokine constructs used in an example embodiment. (B) The purification scheme for obtaining pure, labeled chemokines.

FIG. 13 depicts purification and properties of fluorescently labeled CCL14. (A) Coomassie-stained SDS-PAGE gel. Lane 1: ubiquitin and CCL14-cys after Usp2-cc treatment (sample loaded onto Ni-NTA column), Lanes 2 and 3: Ni-NTA flow-through, Lane 4: Ni-NTA imidazole elution. (B) Representative electrospray mass spectrometry trace. (C) Calcium-mobilization of CCR1-expressing HEK293 cells, to illustrate the purification, cleavage, derivatization and function of fluorescently labeled CCL14.

FIG. 14 depicts CCR1 binding to a chemokine-affinity column. Detergent-solubilized CCR1 was loaded onto a streptavidin column preloaded with biotinylated CCL14. Samples of the column load, flow-through, final wash and column elutions (eluate 1 and eluate 2) were subjected to SDS-PAGE and western blotting analysis using an anti-CCR1 antibody.

FIG. 15 depicts a SPA competition assay. HEK293 membranes, either untransfected (Con) or expressing CCR1 (CCR1) were incubated with increasing concentrations of CCL14, in the presence of either ¹²⁵I-labeled CCL3 (shown as squares or diamonds), or a combination of CCL3-biotin and ¹²⁵I-labeled streptavidin (shown as circles).

FIG. 16 shows protein expression of a 3D3-MIP-1α polypeptide (left) or ub-MIP-1α polypeptide (right). Expression fractions were taken at time 0 and 4 hrs post induction and divided into pellet/insoluble (P) and supernatant/soluble (S) fractions. Full length fusion protein is circled in red and degradation products are indicated with arrows.

FIG. 17 shows protein expression of ub-CCL14 (A) and 3D3-CCL14 (B). Expression fractions were at 4 hrs post induction and divided into pellet/insoluble (P) and supernatant/soluble (S) fractions. Full length fusion protein is circled in red.

FIG. 18 shows protein purification of ub-CXCL8 (H₆-Ub_{WT}-CXCL8) (A) and 3D3-CXCL8 (H₈-Ub_{3D3}-CXCL8) (B). The ub-CXCL8 fusion polypeptide was expressed mostly in a soluble fraction, and was purified over a Ni-NTA column and eluted with increased imidazole. (C) HPLC chromatogram of ub-CXCL8 (H₆-Ub_{WT}-CXCL8) shows misfolded species (circled and indicated with arrow), and (D) HPLC chromatogram of 3D3-CXCL8 shows all protein is correctly folded.

### DETAILED DESCRIPTION

Embodiments of the present invention relate to methods and compositions for high level of expression of fusion proteins. In some embodiments, a fusion protein comprising a modified protein moiety and a protein of interest has a reduced solubility in a host cell compared to the protein of interest. The reduced solubility of the fusion protein increases the yield of the fusion protein.

In some embodiments, a modified protein moiety can include a modified ubiquitin moiety. Ubiquitin is a small eukaryotic protein which offers a natural yield enhancement, and can be removed from a recombinant fusion protein by highly specific proteases, such as deubiquitylating enzymes. Deubiquitylating enzymes do not cleave nonspecific sequences and do not leave additional amino acids at the N terminus of the protein of interest (Baker, R. T. 1996. Protein expression using ubiquitin fusion and cleavage. Curr. Opin. Biotechnol. 7: 541-546; Hondred, D. et al., 1999. Use of ubiquitin fusions to augment protein expression in transgenic plants. Plant Physiol. 119: 713-724). This cleavage occurs precisely after a final glycine residue at the carboxyl terminal of the ubiquitin polypeptide, moreover, this cleavage is irrespective of the amino acid immediately following, with the sole exception of proline, which is cleaved inefficiently (Bachmair, A., Finley, D., and Varshaysky, A. 1986. In vivo half-life of a protein is a function of its amino-terminal residue. Science 234: 179-186).

Ubiquitin fusion protein systems have been used to produce soluble proteins, for example, the pHUE system (Catanzariti, A.-M., T. A. Soboleva, D. A. Jans, P. G. Board, and R. T. Baker, An efficient system for high-level expression and easy purification of authentic recombinant proteins. Protein Sci, 2004. 13(5): p. 1331-1339). Nonetheless, it is often desirable to produce protein in an insoluble form. Accordingly there is a need to produce high levels of recombinant protein in an insoluble form.

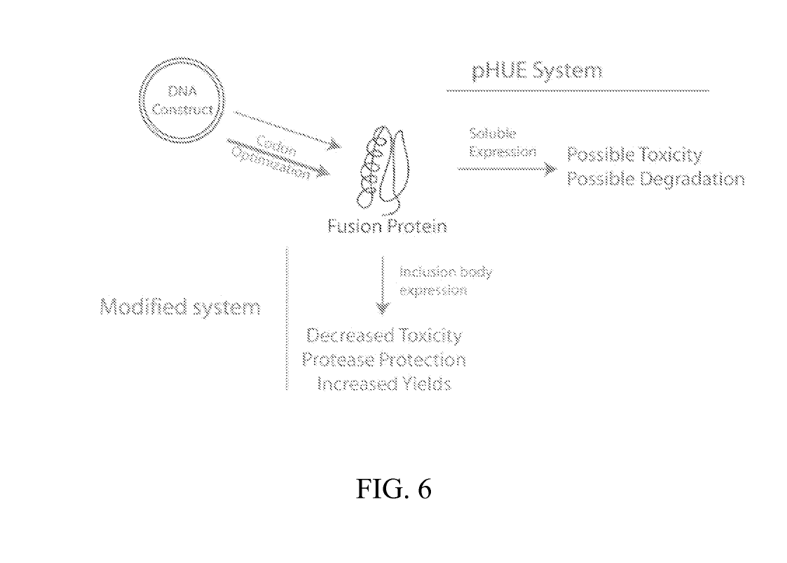

Applicant has discovered the use of particular ubiquitin variants that reduce the solubility of a fusion protein comprising the ubiquitin variants and a protein of interest. The reduced solubility of a fusion protein has several advantages. For example, Applicant has discovered that the yield of particular fusion proteins with reduced solubility is increased. In addition, degradation of fusion proteins with reduced solubility is decreased. Higher yields are obtained with the redesigned core variants because of reduced toxicity and proteolysis due to the “tuned” stability of the ubiquitin fusion (FIG. 6). For example, some proteins, such as chemokines are typically toxic to cells, and sometimes are proteolytically degraded when produced as soluble proteins. In the context of the pHUE system, some chemokines partition into both the soluble and insoluble fraction, making purification more difficult and resulting in lower yields. By redesigning the core amino acids of ubiquitin and altering its stability, fusions can direct chemokines into inclusion bodies. In some embodiments, a specific design can enable proteins to be refolded and efficiently cleaved from the fusion by ubiquitinase, yielding the desired sequence with the precisely desired N-terminus at significantly higher yield than the pHUE system.

Insoluble fusion proteins and fusion proteins with reduced solubility produced by the methods provided herein can be (i) refolded and/or (ii) cleaved with a protease, for example, ubiquitinase, to obtain precisely defined N-termini. Precisely defined N-termini are important for the activity of some proteins, for example, chemokines, where the precise details of the termini defines affinity and activity, namely, whether the chemokines function as antagonists or agonists.

In some embodiments, a protein of interest can include a chemokine Chemokines are small secreted proteins with a mass about 8-12 kDa that function by binding to and signaling through G-protein coupled receptors known as chemokine receptors. Chemokines are divided into four families (CC, CXC, C and CX3C), depending upon the number and location of cysteine residues near the N-terminus. Chemokines are sometimes described as ‘double-edged swords’ in the immune system, as they are necessary for correct function, but are also implicated in many inflammatory diseases, including multiple sclerosis, rheumatoid arthritis and cancer. In addition, two of the chemokine receptors (CXCR4 and CCR5) are necessary for gp120-mediated entry of HIV into cells. Therefore, chemokines and their receptors are attractive targets for pharmaceutical agents.

Since their discovery over 20 years ago, chemokines have been the subject of a large body of research, and multiple structures have been published. However, progress in the field has been slowed by difficulties associated with obtaining sufficient quantities of pure, functional chemokines from heterologous expression systems. The system of choice for chemokine expression is E. coli, due to the cost, ease of use, and ability to isotopically label for NMR studies, and has been successfully used for expression and structure determination of multiple chemokines. However, E. coli expression of chemokines is complicated by the knowledge that in many cases, chemokines are sensitive to small modifications at their N-termini. One well-known example of this is CCL5/RANTES, for which the addition of methionine at the N-terminus (which would occur after expression in E. coli) renders CCL5 inactive as an agonist. Instead, this version, known as Met-RANTES functions as a potent antagonist against both wildtype CCL5 and CCL3 in calcium flux and chemotaxis assays. Some chemokines contain large, bulky residues at position 2 in the sequence, and in previous studies using CCL2 the proline at this position was utilized to specifically remove the initiating methionine residue without affecting the rest of the chemokine. However, this method is obviously dependent upon the chemokine sequence, and so cannot be applied to all chemokines.

An E. coli based expression system may be used whereby the protein of interest is expressed as a fusion to poly-histidine tagged ubiquitin. During purification, a specific deubiquitylating enzyme is added, which removes the N-terminal His-tagged ubiquitin, but leaves the intact chemokine with a native N-terminus. Here this method as a rapid and efficient way to produce milligram quantities of functional chemokines is used. This method has broad applicability to multiple classes of chemokines, and can be used regardless of whether the chemokine is expressed solubly, less solubly, or insolubly in inclusion bodies. Finally, using a destabilizing variant of ubiquitin, it is possible to substantially increase the yield of insoluble protein or protein with reduced solubility, which can subsequently be refolded and purified.

Chemokines and their receptors are important immunomodulatory proteins, and as such are involved in both normal and pathophysiological functions of the immune system. However, chemokines are challenging to study in vitro, as they are often difficult to obtain in milligram quantities in a functional form. This is due to multiple reasons, including low expression levels, difficulty in purification and the importance of a native N-terminus for proper function. This last issue is especially problematic for E. coli expression due to the retention of the N-terminal methionine, which is removed in vivo during secretion. The present disclosure includes the use of a previously reported ubiquitin expression system to express mg/L quantities of multiple chemokines in E. coli, including CCL2/MCP-1, CCL3/MIP-1α, CCL7/MCP-3, CCL13/MCP-4, CCL14/HCC-1, CCL27/CTACK, CCL28/MEC, CXCL8/IL-8, CXCL9/MIG, CXCL10/IP-10 and CXCL11/ITAC. Using some embodiments of the methods described herein, all of the chemokines expressed at levels >1 mg/L. However, a number of the chemokines showed both insoluble and soluble expression, so the use of core-repacked ubiquitin mutants, with lower solubility was explored, to try to increase the final yield. Using the highly soluble CCL7 as a test case, it was shown that these ubiquitin variants caused this chemokine to express at significantly higher levels, as almost exclusively insoluble protein. One particular ubiquitin variant containing I3L, V17L, I23V mutations (3D3), was recognized and efficiently cleaved by a deubiquitylating enzyme after refolding, in a similar manner to wildtype ubiquitin. The ability to use wildtype ubiquitin or ub-3D3 as a fusion system provides a facile method for expressing functional chemokines in quantities appropriate for biophysical and biochemical characterization. Moreover, the ability to rapidly and inexpensively produce labeled chemokines opens the way for their use in many applications, including non-traditional chemokine-receptor interaction studies, both on intact cells and with purified receptor reconstituted in artificial membranes in vitro. Furthermore, the ability to immobilize chemokines to obtain ligand affinity columns aids in efforts to purify chemokine receptors for structural and biophysical studies, by facilitating the separation of functional proteins from their non-functional counterparts.

Chemokines function as extracellular proteins and as such, contain signal sequences that are cleaved during export in vivo. Proper processing of chemokines to obtain a native N-terminus is necessary for function and is therefore an important factor in designing expression systems. When expressed as the native sequence in E. coli, the initiator methionine is often retained resulting in antagonists (e.g. Met-RANTES (Robinson, S. C., et al., A Chemokine Receptor Antagonist Inhibits Experimental Breast Tumor Growth. Cancer Res, 2003. 63(23): p. 8360-8365) and Met-MCP-1 Hemmerich, S., C. et al, Identification of Residues in the Monocyte Chemotactic Protein-1 That Contact the MCP-1 Receptor, CCR2. Biochemistry, 1999. 38(40): p. 13013-13025), similarly chemical modification of N-terminus results in the same effect (e.g. AOP-RANTES Simmons, G., et al., Potent Inhibition of HIV-1 Infectivity in Macrophages and Lymphocytes by a Novel CCR5Antagonist. Science, 1997. 276(5310): p. 276-279). Alternatively, chemokines can be over-processed such that the N-terminal Met along with additional amino acids are removed, again resulting in nonfunctional protein (Paavola, C. D., et al., Monomeric monocyte chemoattractant protein-1 (MCP-1) binds and activates the MCP-1 receptor CCR2B. J Biol Chem, 1998. 273(50): p. 33157-65). In addition, chemokines contain disulfide bonds, which generally cannot be faithfully reproduced in the reducing environment of bacterial cells. For this reason, chemokines are typically expressed as inclusion bodies and then refolded, but there can be difficulties with chemokines prone to high order oligomerization. Because of these problems, many different bacterial systems have been developed for chemokine expression, but for the most part have required optimization on a case by case basis, making production of every chemokine a major project.

Chemokines have been produced using a number of different methods, including chemical synthesis, purification directly from blood, and by expression in mammalian, yeast, insect, and E. coli cells (Paavola, C. D., et al., Monomeric monocyte chemoattractant protein-1 (MCP-1) binds and activates the MCP-1 receptor CCR2B. J Biol Chem, 1998. 273:33157-65; Gong, J. H. et al., Antagonists of monocyte chemoattractant protein 1 identified by modification of functionally critical NH2-terminal residues. J Exp Med, 1995. 181:631-40; Proost, P., et al., Chemical synthesis, purification and folding of the human monocyte chemotactic proteins MCP-2 and MCP-3 into biologically active chemokines Cytokine, 1995. 7:97-104; Harrison, J. K., et al., Mutational analysis of the fractalkine chemokine domain. Basic amino acid residues differentially contribute to CX3CR1 binding, signaling, and cell adhesion. J Biol Chem, 2001. 276:21632-41; Koopmann, W. et al., Identification of a glycosaminoglycan-binding site in chemokine macrophage inflammatory protein-1alpha. J Biol Chem, 1997. 272:10103-9; Masure, S., L. et al., Expression of a human mutant monocyte chemotactic protein 3 in Pichia pastoris and characterization as an MCP-3 receptor antagonist. J Interferon Cytokine Res, 1995. 15(11): p. 955-63; Chakravarty, L., et al., Lysine 58 and histidine 66 at the C-terminal alpha-helix of monocyte chemoattractant protein-1 are essential for glycosaminoglycan binding. J Biol Chem, 1998. 273(45): p. 29641-7; Polo, S., et al., Enhancement of the HIV-1 inhibitory activity of RANTES by modification of the N-terminal region: dissociation from CCR5 activation. Eur J Immunol, 2000. 30(11): p. 3190-8; Mayer, M. R. et al., Identification of receptor binding and activation determinants in the N-terminal and N-loop regions of the CC chemokine eotaxin. J Biol Chem, 2001. 276(17): p. 13911-6; Myers, J. A., et al., Expression and purification of active recombinant platelet factor 4 from a cleavable fusion protein. Protein Expr Purif, 1991. 2(2-3): p. 136-43; Van Coillie, E., et al., Functional comparison of two human monocyte chemotactic protein-2 isoforms, role of the amino-terminal pyroglutamic acid and processing by CD26/dipeptidyl peptidase IV. Biochemistry, 1998. 37(36): p. 12672-80; Wain, J. H., et al., Rapid site-directed mutagenesis of chemokines and their purification from a bacterial expression system. J Immunol Methods, 2003. 279(1-2): p. 233-49; Ye, J., et al., Characterization of binding between the chemokine eotaxin and peptides derived from the chemokine receptor CCR3. J Biol Chem, 2000. 275(35): p. 27250-7). Some of these systems are impractical based on the cost of production (chemical synthesis) or the final protein yield (purification from blood). Of the remaining systems, the most attractive is E. coli expression due to its ease, low cost, high protein yields, and ability to produce chemically (isotopic or selenomethionine) labeled protein.

The fact that chemokines are secreted proteins provides two problems that must be overcome. Like most secreted proteins, chemokines contain intramolecular disulfide bonds. The reducing environment of the cytosol is not conducive to the production of disulfides. This can be overcome by secreting the protein, either into the media, in mammalian/yeast/insect cell expression, or into to the periplasm, in E. coli. These methods tend to produce lower protein yields, and so cytosolic expression is preferred. A number of chemokines have been expressed and purified from E. coli supernatants (Mayer, M. R. et al., Identification of receptor binding and activation determinants in the N-terminal and N-loop regions of the CC chemokine eotaxin. J Biol Chem, 2001. 276(17): p. 13911-6; Ye, J., et al., Characterization of binding between the chemokine eotaxin and peptides derived from the chemokine receptor CCR3. J Biol Chem, 2000. 275(35): p. 27250-7), while other chemokines (e.g. like CCL2), can be made to express solubly using thioredoxin deficient cell lines like TAP-302 (Lau, E. K., et al., Identification of the Glycosaminoglycan Binding Site of the CC Chemokine, MCP-1: IMPLICATIONS FOR STRUCTURE AND FUNCTION IN VIVO. J. Biol. Chem., 2004. 279(21): p. 22294-22305) and/or by decreasing the temperature at which the protein is expressed. However, many chemokines express partially or exclusively insolubly in E. coli, although their small size and presence of disulfides makes them relatively easy to refold. As a result a number of chemokines have been purified from inclusion bodies and have been shown to be properly refolded and active (Wain, J. H., et al., Rapid site-directed mutagenesis of chemokines and their purification from a bacterial expression system. J Immunol Methods, 2003. 279(1-2): p. 233-49; Proudfoot, A. E., et al., Extension of recombinant human RANTES by the retention of the initiating methionine produces a potent antagonist. J Biol Chem, 1996. 271(5): p. 2599-603).

The second major problem with chemokine expression comes from the fact that in vivo they contain a signal peptide that gets cleaved off during their secretion, thereby removing the initiator methionine. Conversely, non-secreted expression of chemokines results in the presence of the initiator methionine at the N-terminus. Research has shown that the presence of an N-terminal methionine often affects the action of chemokines and turns them into powerful antagonists (Proudfoot, A. E., et al., Extension of recombinant human RANTES by the retention of the initiating methionine produces a potent antagonist. J Biol Chem, 1996. 271(5): p. 2599-603). This is not surprising since the N-terminus of chemokines has been shown to be critical for binding and signaling (Paavola, C. D., et al., Monomeric monocyte chemoattractant protein-1 (MCP-1) binds and activates the MCP-1 receptor CCR2B. J Biol Chem, 1998. 273(50): p. 33157-65). It is therefore necessary to remove this residue to produce active protein. N-terminal tags that are commonly used to simplify purification also have to be removed. Aminopeptidase can be used to remove residues from the N-terminus, but it will nonspecifically remove residues until it encounters a proline. This method has been used to produce active CCL2, which fortuitously has a proline at position 2, but cannot be used for all chemokines (Lau, E. K., et al., Identification of the Glycosaminoglycan Binding Site of the CC Chemokine, MCP-1: IMPLICATIONS FOR STRUCTURE AND FUNCTION IN VIVO. J. Biol. Chem., 2004. 279(21): p. 22294-22305). For those chemokines that do not have a proline at the second position, protease cleavage can be used to produce the native N-terminus. Thrombin, enterokinase, and factor-X protease have all been used to produce properly cleaved products (Mayer, M. R. et al., Identification of receptor binding and activation determinants in the N-terminal and N-loop regions of the CC chemokine eotaxin. J Biol Chem, 2001. 276(17): p. 13911-6; Ye, J., et al., Characterization of binding between the chemokine eotaxin and peptides derived from the chemokine receptor CCR3. J Biol Chem, 2000. 275(35): p. 27250-7). The problem with using these proteases is that they are not always specific, and so in many cases these proteases will also cleave at additional sites within the protein producing unwanted truncations.

Fusion proteins are routinely used to increase the yield of proteins, increase solubility or create specific cleavage sites for proteins during over-expression. One example of a fusion system is the Baker ubiquitin/ubiquitinase system (the pHUE system) (Catanzariti, A.-M., et al., An efficient system for high-level expression and easy purification of authentic recombinant proteins. Protein Sci, 2004. 13:1331-1339).

Described herein are embodiments which include systems for producing a wide range of fusion proteins, including chemokine fusion proteins, with high levels of expression. Structural and biochemical studies of the chemokine binding proteins and their interactions with chemokines require a cheap and reliable source of chemokines. At present, most chemokines can be commercially purchased, but the cost of obtaining sufficient quantities makes their use in structural studies prohibitive. It is therefore necessary to develop an expression system to produce the chemokines in-house at a lower cost.

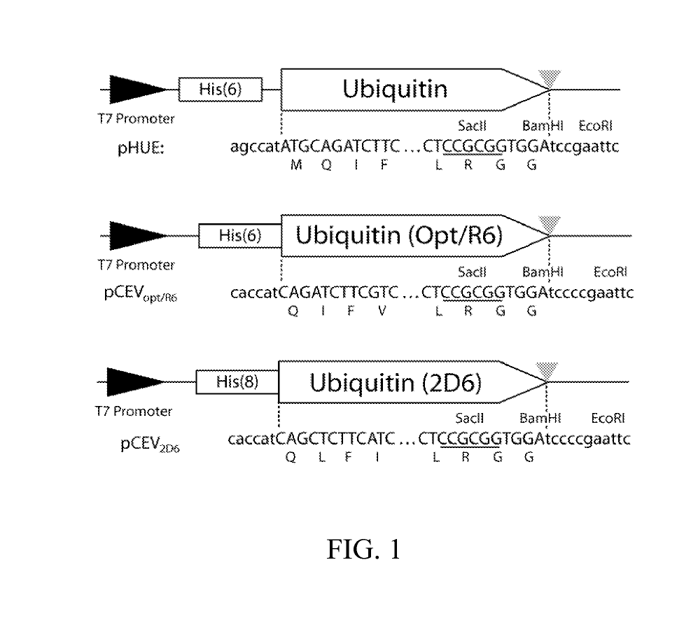

The pHUE expression/cleavage system can be used to produce a very specific cleavage site described in Catanzariti, A.-M., T et al., An efficient system for high-level expression and easy purification of authentic recombinant proteins. Protein Sci, 2004. 13(5): p. 1331-1339, hereby incorporated by reference for its discussion on material and methods of the pHUE system. Briefly, the system involves fusing His6-ubiquitin to the N-terminus of a protein. The tagged ubiquitin can be removed from the protein through the use of ubiquitinase, which results in a very specific cleavage after the second glycine at the C-terminus of ubiquitin (FIG. 1). However, using the pHUE system, most chemokines are expressed with high variability. Moreover, particular chemokines, for example, CCL3, shows no expression using the pHUE system.

Expression and purification of large quantities of active chemokines for biochemical experiments has proved to be difficult thus far. The requirement for properly formed disulfides and an exact N-terminus has limited the range of chemokines that can be expressed at high levels. The pHUE system has increased the range of chemokines that can be expressed and purified from E. coli. However, there still remain chemokines that either express at low levels or are rapidly degraded.

To overcome this, the pHUE system was modified by altering the ubiquitin codon sequence. Three different ubiquitin sequences were used: one that was merely codon optimized and two where the amino acid sequences were both computationally designed and codon optimized. Computationally designed ubiquitin sequences are core-redesigns produced using the Repacking of Cores (ROC) program (Lazar, G. A., et al., De novo design of the hydrophobic core of ubiquitin. Protein Sci, 1997. 6(6): p. 1167-1178).

Codon optimization of ubiquitin had little effect on the expression levels of the fusion proteins. There was some increase in expression of some insoluble chemokine fusions, but it was only minimal. In the case of solubly expressing fusions, there was either a small increase in the solubly expressed protein, CXCL11 and CCL3, or an increase in the insoluble fraction, CCL7. This result is not too surprising, as some of the non-codon optimized fusions (e.g. CCL7) express extremely well, and suggests that the rate of expression of ubiquitin is not the limiting factor, but rather that it is the solubility of the fusion protein coupled to the chemokine itself that is the limiting factor. CCL3 expressed at higher levels, but was still subject to degradation which resulted in no net increase of protein. When CCL7 was expressed with wildtype ubiquitin, there was a small amount of protein that was found to be in inclusion bodies which increased over time. The codon optimized CCL7 construct resulted in faster inclusion body production rather than any increase in soluble expression.

In an attempt to change the solubility of the ubiquitin-chemokine fusion proteins, two core redesigned ubiquitin amino acid sequences were used. The two sequences used, 2D6 and R6, were less stable, ΔΔG=−3.2 and −4.5 kcal/mol respectively, than wildtype and express insolubly. These two sequences were chosen because they were they most unstable mutants that could still be refolded. Expression tests showed that all chemokines fused to either 2D6 or R6 expressed insolubly, with no trace of soluble expression. The expression levels of all five chemokines tested higher than those seen with the codon optimized or wildtype constructs. Additionally, no degradation was observed in the inclusion bodies, suggesting that these designed constructs will be useful for expressing other chemokines susceptible to degradation. Although refolding studies of these fusions have not yet been undertaken, this should be relatively simple, as 2D6, R6, and all of the pHUE ubiquitin chemokine-fusions tested to date have been refolded successfully.

The use of the core-redesigned ubiquitin sequences produce the largest increase in protein yield of a number of different chemokines. In addition, the expression is universally insoluble which will protect the protein from possible degradation. Purifying the fusion under denaturing conditions will avoid the issue seen recently in the lab where the ubiquitin-CCL2 fusion precipitated on the column during purification. The chemokines used in this study display a wide range of expression profiles in the pHUE system, and they all could be successfully expressed in high levels using the core-redesigned (2D6 and R6) ubiquitin systems. This indicates that these new expression vectors are generally applicable for chemokine expression and can be used to boost the expression of difficult to produce chemokines.

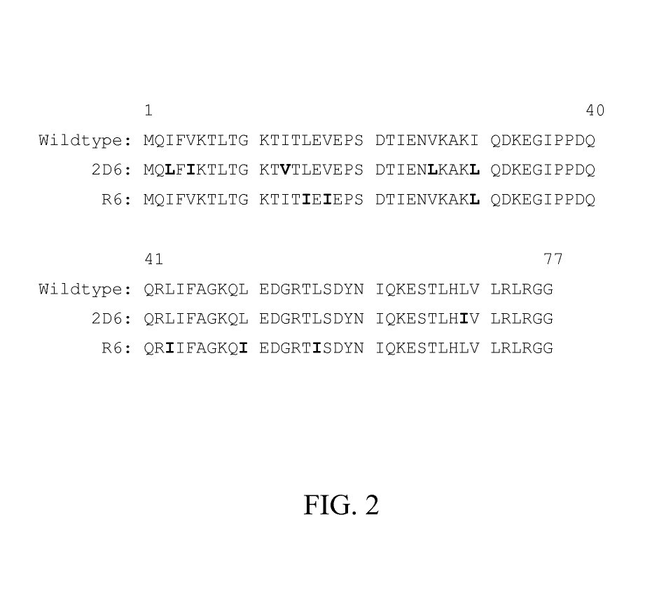

To increase expression yields, as well as to make the ubiquitin fusion system more generally applicable to chemokines, the pHUE system was modified. Most of the DNA sequences of chemokines for E. coli expression were codon-optimized to increase their expression yields. Codon-optimization of ubiquitin-chemokine fusions were performed to increase levels of expression of said ubiquitin-chemokine fusions. Insoluble ubiquitin core-redesigns that can be refolded include those shown in FIG. 2 (Lazar, G. A., et al., De novo design of the hydrophobic core of ubiquitin. Protein Sci, 1997. 6(6): p. 1167-1178).

The following examples include descriptions of the stability and expression yields of chemokines expressed as insoluble fusion proteins or fusion proteins with reduced solubility. However, it will be appreciated that the methods and compositions described herein may be utilized with proteins of interest other than chemokines, including proteins of interest which are toxic or which provide lower yields using alternative expression systems. In particular, expression vectors and their expression profiles are described, where the replacement of wildtype ubiquitin with a core redesigned ubiquitin fusion partner had lower stability, shifting expression to inclusion bodies. Such less stable fusions were expressed at higher levels than wildtype or codon optimized versions of ubiquitin.

### Methods for Producing Recombinant Proteins

Some embodiments of the methods and compositions provided herein relate to methods for producing a recombinant protein. In some embodiments, a recombinant protein can be produced by expressing a fusion protein in a host cell. In some embodiments, a fusion protein comprises a modified protein moiety and a protein of interest. The fusion protein can be expressed from a single polynucleotide sequence in a host cell. Examples of host cells include eukaryotic cells, such as mammalian cells, insect cells, plant cells, yeast, and prokaryotic cells, such as E. coli. In particular embodiments, an expressed fusion protein comprising a protein of interest has a reduced solubility in a host cell compared to the solubility of the protein of interest expressed in the host cell. In some such embodiments, the expressed fusion protein can be enriched in inclusion bodies of the host cell.

Polynucleotides encoding a fusion protein can be constructed by methods well known in the art. A polynucleotide encoding a fusion protein can include nucleotide sequences for increasing the expression of the fusion protein in a host cell. Such nucleotide sequences include promoters, enhancers, and terminal sequences. Generally, a polynucleotide encoding a fusion protein can include a sequence encoding a 5′ modified protein moiety, a 3′ protein of interest, and a protease recognition cleavage site therebetween. A protease recognition cleavage site can be used to cleave a protein of interest from an expressed fusion protein. Examples of proteases that can be used with the methods and compositions provided herein include ubiquitinases, such as Usp2-cc. In some embodiments, a polynucleotide encoding a fusion protein can include sequences to facilitate purification of a fusion protein. An example includes a sequence encoding a plurality of Histidine residues, useful to purify a fusion protein using a Nickel column.

More embodiments include providing conditions for said protein of interest to refold into an active form. In some such embodiments the protein of interest is subsequently cleaved from the modified protein moiety. Conditions for said protein of interest to refold into an active form can be determined by methods well known in the art.

### Modified Protein Moieties

Some embodiments of the methods and compositions provided herein relate to modified protein moieties. In some embodiments, a modified protein moiety includes a polypeptide sequence that reduces the solubility of an expressed fusion protein comprising a protein of interest in a cell compared to the solubility of the protein of interest in the cell. Such modified protein polypeptides can be designed de novo by methods well known in the art (See e.g., DeGrado W. F., et al., (1999) Annu Rev. Biochem. 68:779-819).

Examples of modified protein moieties include modified ubiquitin polypeptides (Lazar G. A. et al., (1997) Protein Sci. 6:1167-1178). In some embodiments, the modified ubiquitin moiety comprises an increased frequency or number of hydrophobic amino acid residues compared to a wild type ubiquitin moiety. In some embodiments, the modified ubiquitin moiety comprises a decreased frequency or number of hydrophobic amino acid residues compared to a wild type ubiquitin moiety. Examples of modified ubiquitin moieties include ubiquitin sequences with one or more mutations at positions selected from the group consisting of I3, V5, I13, L15, V17, I23, V26, I30, L43, L50, L56, and L69, relative to a wild type ubiquitin sequence such as SEQ ID NO:09 (FIG. 2). In some embodiments, a modified ubiquitin moiety comprises a ubiquitin sequence with one or more mutations selected from the group consisting of I3L, I13V, V5I, L15I, V17L, I23V, V26L, I30L, L43I, L50I, L56I, and L69I, relative to a wild type ubiquitin sequence such as SEQ ID NO:09. In particular embodiments, a modified ubiquitin moiety comprises a ubiquitin sequence with one or more mutations selected from the group consisting of I3L, V17L, and I23V, relative to a wild type ubiquitin sequence such as SEQ ID NO:09. In particular embodiments, a modified ubiquitin moiety comprises an ubiquitin sequence with one or more mutations selected from the group consisting of I3L, V5I, I13V, V26L, I30L, and L69I, relative to a wild type ubiquitin sequence such as SEQ ID NO:09. In particular embodiments a modified ubiquitin moiety comprises an ubiquitin sequence with one or more mutations selected from the group consisting of L15I, V17I, I30L, L43I, L50I, and L56I, relative to a wild type ubiquitin sequence such as SEQ ID NO:09. In some embodiments, a modified ubiquitin moiety comprises a sequence selected from the groups consisting of SEQ ID NO:10, SEQ ID NO:11, and SEQ ID NO:26. In certain embodiments, a modified ubiquitin moiety comprises SEQ ID NO:26.

### Proteins of Interest

Some embodiments of the methods and compositions provided herein relate to proteins of interest. Generally, a protein of interest can include any polypeptide. In certain embodiments, a protein of interest can be soluble, insoluble or partially soluble in a cell in which it is expressed. In preferred embodiments, the protein of interest is soluble.

The methods and compositions provided herein include expression of a fusion protein comprising a modified protein moiety and a protein of interest with reduced solubility in a cell compared to the solubility of the protein of interest in the cell. In some embodiments, such methods and compositions are advantageously useful for the production of proteins of interest that would be toxic to a cell in which it is expressed in a soluble form. In some embodiments, such methods and compositions are also advantageously useful for the production of proteins of interest that would be degraded in a cell in which it is expressed in a soluble form. Therefore, in some embodiments, a protein of interest includes a protein toxic to a cell in which it is expressed, in which the protein of interest is in soluble form. In addition, in some embodiments, a protein of interest includes a protein that is degraded in a cell in which it is expressed, in which the protein of interest is in soluble form.

In certain embodiments, a protein of interest can include a chemokine or a cytokine Examples of such proteins of interest include: CCL2 (SEQ ID NO:27), CCL3 (SEQ ID NO:28), CCL7 (SEQ ID NO:29), CCL13 (SEQ ID NO:30), CCL14 (SEQ ID NO:31), CCL27 (SEQ ID NO:32), CCL28 (SEQ ID NO:33), CXCL8 (SEQ ID NO:34), CXCL9 (SEQ ID NO:35), CXCL10 (SEQ ID NO:36), CXCL11 (SEQ ID NO:37), CCL14 (SEQ ID NO:38), CXCL1 (SEQ ID NO:39), CXCL2 (SEQ ID NO:40), CXCL3 (SEQ ID NO:41), CXCL4 (SEQ ID NO:42), CXCL5 (SEQ ID NO:43), CXCL6 (SEQ ID NO:44), CXCL7 (SEQ ID NO:45), CXCL12 (SEQ ID NO:46), CXCL13 (SEQ ID NO:47), CXCL14 (SEQ ID NO:48), CXCL16 (SEQ ID NO:49), XCL1 (SEQ ID NO:50), XCL2 (SEQ ID NO:51), CX3CL1 (SEQ ID NO:52), CCL1 (SEQ ID NO:53), CCL4 (SEQ ID NO:54), CCL5 (SEQ ID NO:55), CCL8 (SEQ ID NO:56), CCL11 (SEQ ID NO:57), CCL15 (SEQ ID NO:58), CCL16 (SEQ ID NO:59), CCL17 (SEQ ID NO:60), CCL18 (SEQ ID NO:61), CCL19 (SEQ ID NO:62), CCL20 (SEQ ID NO:63), CCL21 (SEQ ID NO:64), CCL22 (SEQ ID NO:65), CCL23 (SEQ ID NO:66), CCL24 (SEQ ID NO:67), CCL25 (SEQ ID NO:68), and CCL26 (SEQ ID NO:69).

In particular embodiments, a nucleic acid encoding a protein of interest can be codon-optimized for expression in a particular cell. Methods for codon optimization to achieve optimal expression in a particular organism, such as E. coli and yeast, are well known in the art.

### EXAMPLES

### Example 1

### Wild-Type Ubiquitin-Chemokine Expression

Expression tests of twenty ubiquitin-chemokine fusion constructs (five different chemokines with four versions of ubiquitin) were undertaken. Table 1 shows expression of pHUE/pCEV ubiquitin chemokine fusions.

TABLE 1

| Vector | | | Soluble | Insoluble |
|---|---|---|---|---|
| Name | Backbone | Chemokine | expression | Expression |
| pCEV-01 | pHUE | CXCL11 | trace | ++++ |
| pCEV-02 | pHUE | CCL3 | + | ++ |
| pCEV-03 | pHUE | CCL28 | + | +++ |
| pCEV-04 | pHUE | CCL7 | +++ | trace |
| pCEV-05 | pHUE | CCL13 | − | +++ |
| pCEV-06 | pCEV_{Opt}-27 | CXCL11 | + | ++++ |
| pCEV-07 | pCEV_{Opt}-27 | CCL3 | ++ | +++ |
| pCEV-08 | pCEV_{Opt}-27 | CCL28 | − | +++ |
| pCEV-09 | pCEV_{Opt}-27 | CCL7 | +++ | + |
| pCEV-10 | pCEV_{Opt}-27 | CCL13 | − | ++++ |
| pCEV-11 | pCEV_{2D6}-27 | CXCL11 | − | ++++ |
| pCEV-12 | pCEV_{2D6}-27 | CCL3 | − | +++++ |
| pCEV-13 | pCEV_{2D6}-27 | CCL28 | − | +++ |
| pCEV-14 | pCEV_{2D6}-27 | CCL7 | − | +++++ |
| pCEV-15 | pCEV_{2D6}-27 | CCL13 | − | ++++ |
| pCEV-16 | pCEV_{R6}-27 | CXCL11 | − | ++++ |
| pCEV-17 | pCEV_{R6}-27 | CCL3 | − | +++++ |
| pCEV-18 | pCEV_{R6}-27 | CCL28 | − | +++ |
| pCEV-19 | pCEV_{R6}-27 | CCL7 | − | +++++ |
| pCEV-20 | pCEV_{R6}-27 | CCL13 | − | +++++ |

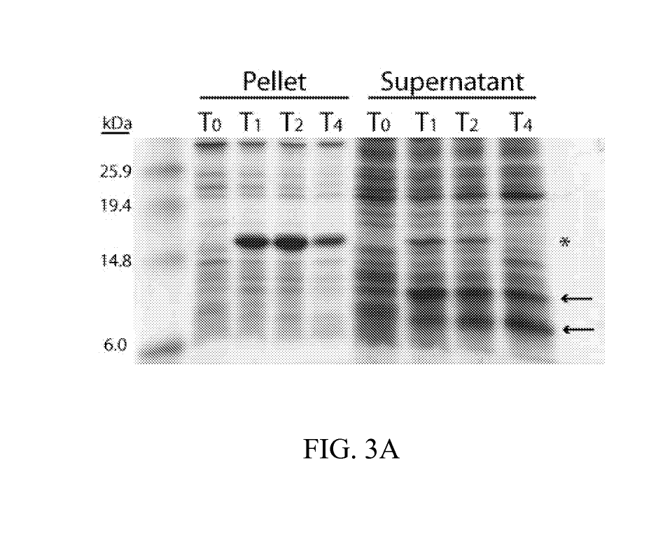

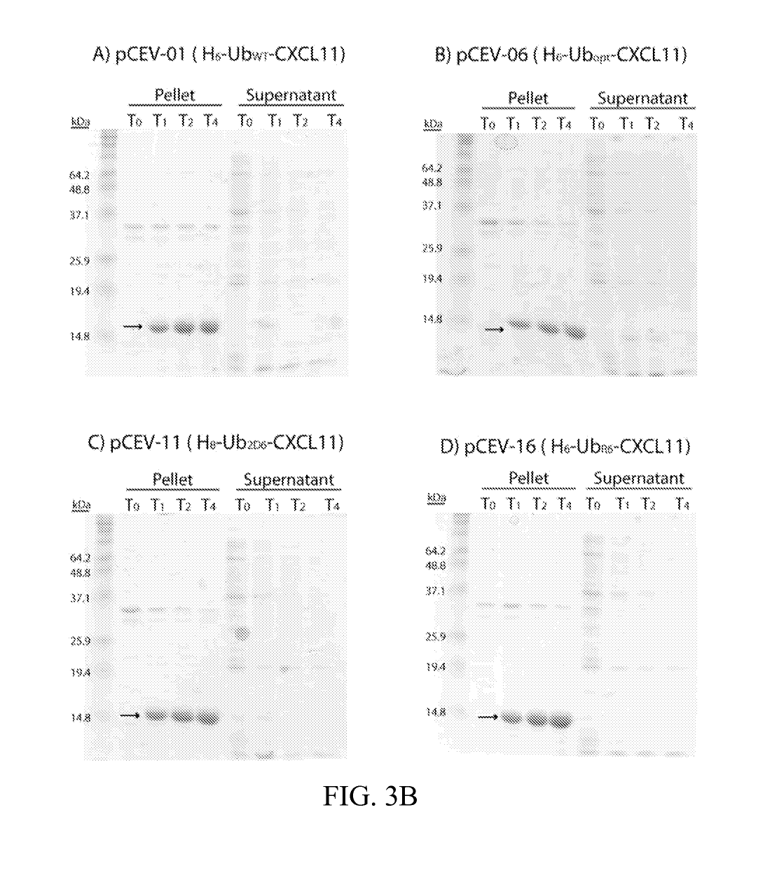

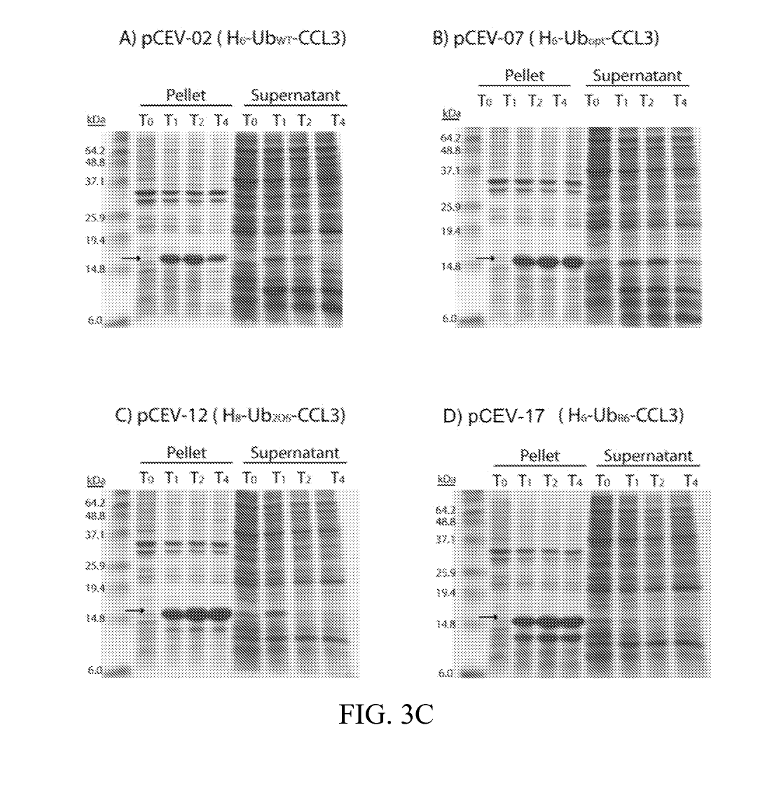

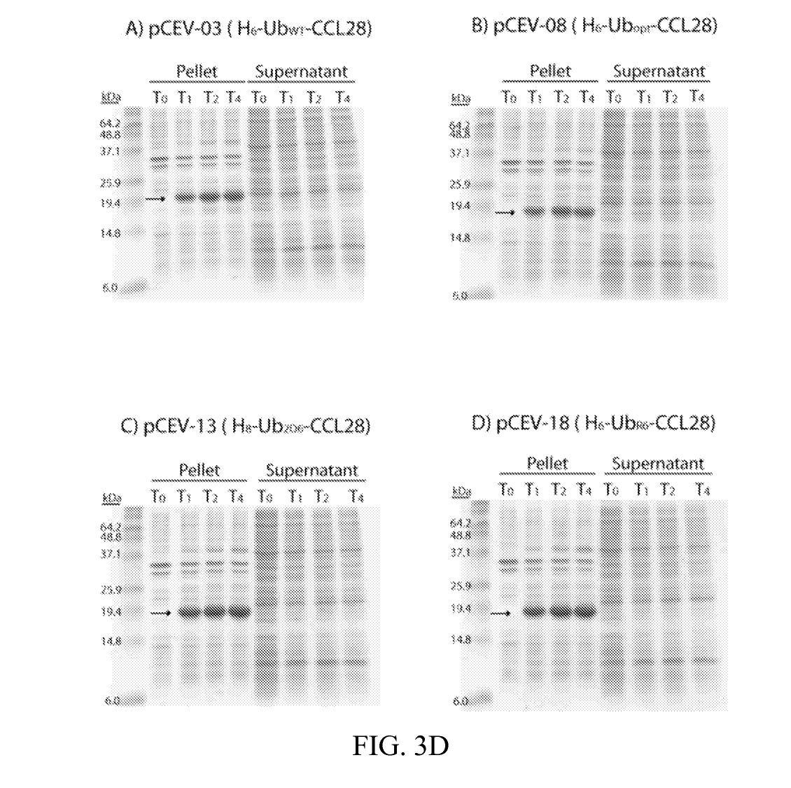

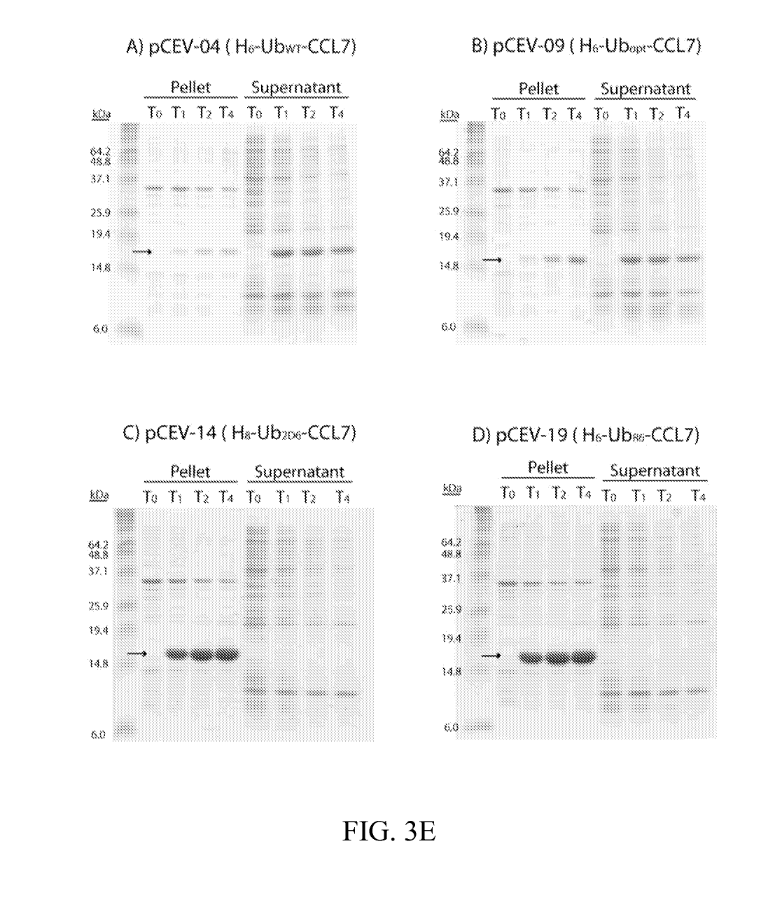

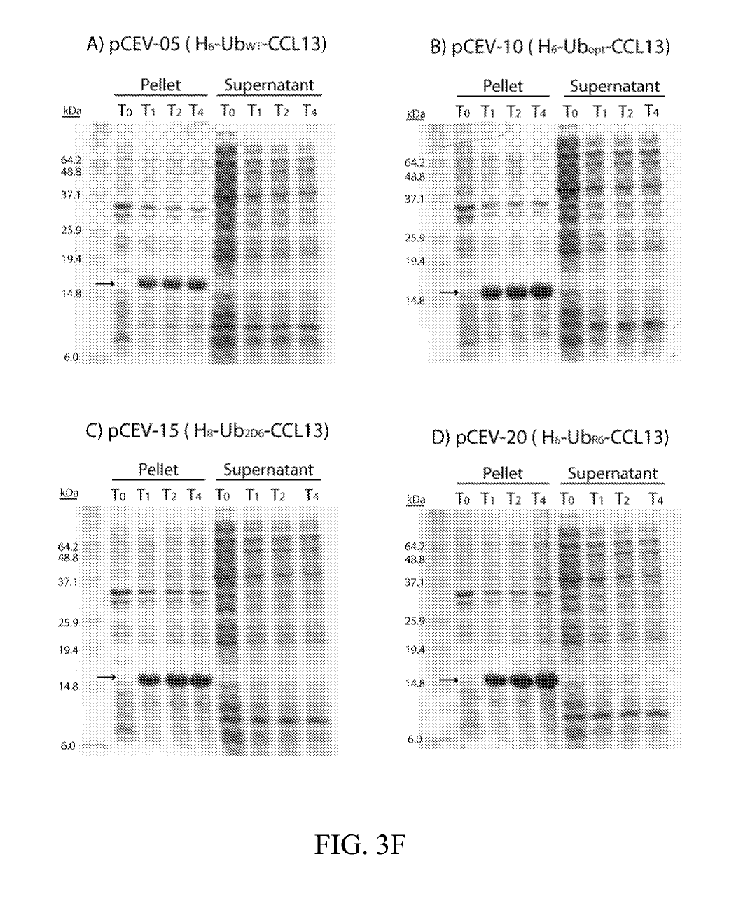

CXCL11, CCL7 and CCL28 showed at least small amounts of soluble expression in the pHUE system, while CCL13 expressed insolubly. In contrast, CCL3 showed a reasonable level of insoluble expression, but very little full-length soluble protein. However, a number of smaller molecular weight bands, presumably truncation products, were observed (FIGS. 3A-3F).

### Example 2

### Codon-Optimized Ubiquitin-Chemokine Expression

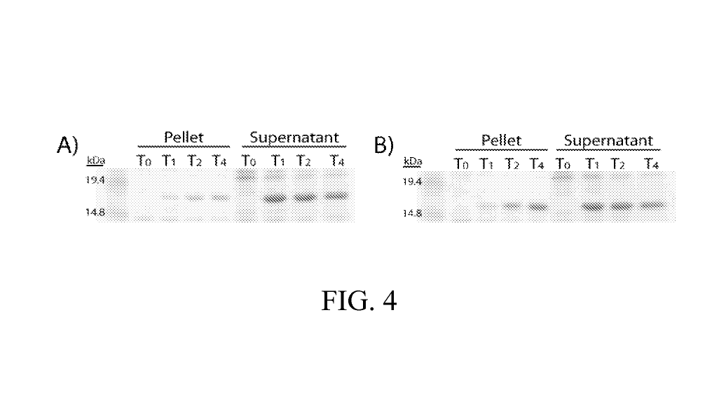

To increase the yield of ubiquitin-fused chemokines, the codon sequence for ubiquitin was optimized based on E. coli codon usage. The expression patterns of the codon-optimized ubiquitin fusion constructs were similar to those seen with pHUE. Optimization of ubiquitin did not change the solubility of any of the fusions, but did affect the overall expression levels in a number of cases. Increased soluble expression was only seen in the lowest level expressers, CXCL11 and CCL3. Soluble CXCL11 expression increased slightly, but the protein was rapidly degraded, while in the case of CCL3, there was a noticeable increase in expression of both full length protein and truncation products. Conversely, codon-optimization of ubiquitin resulted in a slight decrease in soluble expression of CCL28. CCL7 and CCL13 both show increased insoluble expression and no change in soluble expression (FIG. 4). When CCL7 was expressed in pHUE, a small amount of insoluble expression was seen which increased over time. This suggests that over time the protein is becoming too concentrated and therefore aggregates to form inclusion bodies. As a result, the increased expression levels of the codon optimized sequence lead to faster inclusion body formation without any increase in the soluble protein.

### Example 3

### Expression of 2D6 and R6 Mutants of Ubiquitin as Chemokine Fusions

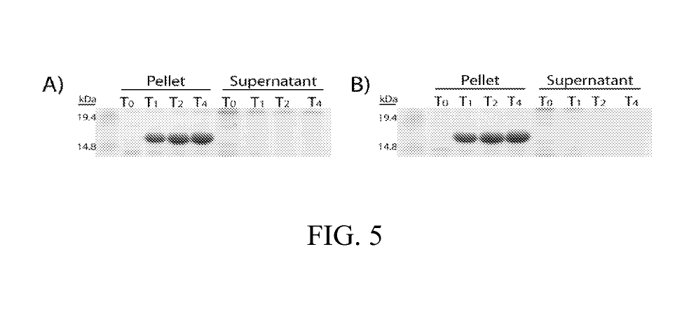

To deal with heterogonous expression and degradation problems, the amino acid sequence of ubiquitin was altered. Computational designs where the ubiquitin core was repacked led to the generation of a number of altered ubiquitin sequences which were less stable than wildtype (Paavola, C. D., et al., Monomeric monocyte chemoattractant protein-1 (MCP-1) binds and activates the MCP-1 receptor CCR2B. J Biol Chem, 1998. 273(50): p. 33157-65). Two of those sequences, 2D6 and R6, were fused to chemokines via the pHUE system. All chemokine fusions expressed using pCEV_{2D6} and pCEV_{R6} resulted in insoluble expression. In all cases the expression levels were higher than those seen for the wildtype (pHUE) or codon optimized (pCEVₒₚₜ) versions of ubiquitin (FIG. 5).

### Example 4

### Codon-Optimization Sequences

Codon-optimization sequences for human CCL3, CCL13, CCL28, and CXCL11 for expression in E. coli were generated using computer program which replaces the natural codons of a given protein with codons observed at high frequency in bacterial cells. Repetitive sequences, high GC content, and mRNA structure are then eliminated with cycles of Monte Carlo optimization.

### Example 5

### Vector Cloning

Six pCEV vectors were designed to be compatible with pHUE chemokine vectors. The inserts were designed to have Met-Gly-Ser-Ser (SEQ ID NO:12) sequence followed by either a (His)₆ (SEQ ID NO:13) or a (His)₈ (SEQ ID NO:14) at the N-terminus of ubiquitin and use a similar C-terminal sequence that contains a SacII sequence (FIG. 1). Using SacII at the end of ubiquitin allows the rapid production of expression constructs via sub-cloning from the previous pHUE-based chemokine expression vectors. Codon optimized (Ubₒₚₜ) and R6 (Ub_{R6}) ubiquitin were produced using the following primers (MWG Biotech, High Point, N.C.) which resulted in a nucleic acid encoding a (His)₆ at the N-terminus:

(SEQ ID NO: 15)
Fwd:
5′-
CTTGATAGCCATATGGGCTCTTCCCACCATCACCATCACCATCAGATCTT
CGTCAAGACGTTAACC-3′;
and
(SEQ ID NO: 16)
Rev:
5′-
GCGATAGAATTCGGATCCACCGCGGAGACGTAAGACAAGATGTAAGGTCG
ACTCC-3′.

The 2D6 variant of ubiquitin (Ub_{2D6}) was made using the following primers which resulted in a nucleic acid encoding (His)₈ at the N-terminus:

(SEQ ID NO: 17)
Fwd:
5′-
CTTGATAGCCATATGGGCTCTTCCCACCATCACCATCACCATCACCATCA
GCTCTTCATCAAGACGTTAACC -3′;
and
(SEQ ID NO: 18)
Rev:
5′-
GCGATAGAATTCGGATCCACCGCGGAGACGTAAGACAATATGTAAGGTCG
ACTCC-3′.

All modified ubiquitin fragments were first digested with NdeI and EcoRI, after which they were ligated into pET27b and pSV212 using the same restriction sites. pSV212 is a pET-21 derived expression vector that contains the same promoter/RBS region as pET-27b, but contains ampicillin resistance instead of a kanamycin resistance.

CCL3 (MIP-1a), CCL7 (MCP-3), CCL13 (MCP-4), CCL28 (MEC) and CXCL11 (1-TAC) were subcloned from respective pHUE based-vectors into pCEVOpt-27, pCEV2D6-27 and pCEVR6-27 using SacII and HindIII. Table 2 lists the names and genes of constructs made.

TABLE 2

| | | | Resistance |
|---|---|---|---|
| Vector Name | N-term Sequence | Ubiquitin | Gene |
| pCEVₒₚₜ-21 | MGSS-(His)₆ | codon optimized | ampicillin |
| pCEVₒₚₜ-27 | MGSS-(His)₆ | codon optimized | kanamycin |
| pCEV_{2D6}-21 | MGSS-(His)₈ | designed - 2D6 | ampicillin |
| pCEV_{2D6}-27 | MGSS-(His)₈ | designed - 2D6 | kanamycin |
| pCEV_{R6}-21 | MGSS-(His)₆ | designed - R6 | ampicillin |
| pCEV_{R6}-27 | MGSS-(His)₆ | designed - R6 | kanamycin |

### Example 6

### Expression Tests

All constructs were transformed into BL21(DE3)pLysS cells and plated on LB plates containing chloramphenicol and either carbenicillin (pHUE) or kanamycin (pCEV-27 based vectors). Once single colonies were visible, a few were picked and transferred to 50 ml of LB+antibiotic and grown overnight at 37° C. 100 ml samples of LB+antibiotics in baffled flasks were inoculated to an OD₆₀₀ₙₘ=0.05 the following morning. Cultures were grown at 37° C. with shaking (200 rpm). OD₆₀₀ₙₘ time points were taken every 30-60 min until the OD₆₀₀ₙₘ was between 0.5 and 0.6, at which time a 1 ml sample was taken and the rest of the culture induced with 1 mM IPTG. 1 ml samples were taken at time points of 1, 2 and 4 hrs post induction. All 1 ml samples were treated as follows: samples were spun down at 16,000×g in a tabletop microfuge (eppendorf 5415 c) for 1 minute. Excess media was removed and the cell pellet was resuspended in 50 mM Tris 8.0, 300 mM NaCl, 5 mM MgSO₄, 0.1% Tween-20 at a volume of 100 μl of buffer/1 ODU₆₀₀ₙₘ. 1 μl of 1 mg/ml DNase and 1 μl of 1 mg/ml lysozyme were added to each sample prior to freezing at −20° C. To assay the levels of soluble and insoluble expression the samples were run on 15% SDS-PAGE gels. Protein bands were visualized using Coomassie stain.

### Example 7

### Protein Yields

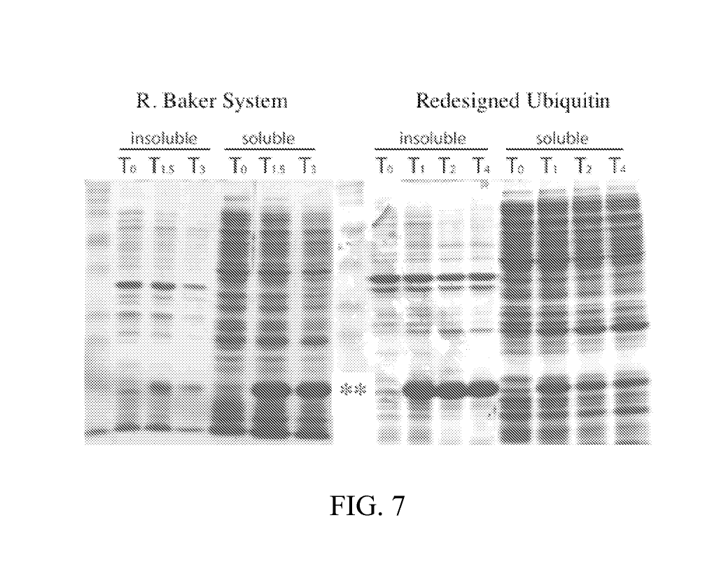

Table 3 shows a comparison of the estimated yields (mg/L) of chemokine from the pHUE system, compared to yields with redesigned ubiquitin. The redesigned ubiquitin can be used to shift protein from the soluble to the insoluble fraction (FIG. 7).

TABLE 3

| | Baker | |
|---|---|---|
| Chemokine | system | Redesigned Ubiquitin |
| ITAC | 2.5 | 16.47 |
| MCP-4 | 3.5 | 16.86 |
| MEC | 8 | 20.47 |
| MIP-1α | No | 18.43 |
| | expression | |
| MCP-1 | | 9.97 |
| IL-8 | | 6.53 |
| MIG | 5 | 40.84 |
| IP-10 | 20 | 36.64 |

### Example 8

### Using Ubiquitin to Rapidly Produce Functional Chemokines

### Materials and Methods:

Materials: pHUE and pUsp2-cc vectors contained inserts for wildtype ubiquitin and the mouse deubiquitinylating enzyme Usp2, respectively.

### Expression and Purification of Ubiquitin-Chemokine Fusions

DNA sequences encoding human CCL2, CCL3 CCL7, CCL13, CCL14, CCL27, CCL28, CXCL8, CXCL9, CXCL10 and CXCL11 were codon-optimized for E. coli using an in-house program and constructed using overlapping primer synthesis. Table 4 provides examples of vectors with chemokine sequences inserted therein.

TABLE 4

| | |
|---|---|
| 3D3-MIP 1β WT | ATGGGCTCTTCCCACCATCACCATCACCATCACCATCAGCTC |
| Features: | TTCGTCAAGACGTTAACCGGTAAAACCATAACTCTAGAACT |
| (1) His TAG | TGAACCATCCGATACCGTCGAAAACGTTAAGGCTAAAATTC |
| (double- | AAGACAAGGAAGGTATTCCACCTGATCAACAACGTTTGATC |
| underlined); | TTTGCCGGTAAGCAGCTCGAAGACGGTCGTACGCTGTCTGA |
| (2) Ubiquitin | TTACAACATTCAGAAGGAGTCGACCTTACATCTTGTCTTAC |
| encoding region | GTCTCCGCGGTGGAGCACCAATGGGCTCTGACCCTCCCACC |
| (underlined). | GCCTGCTGCTTTTCTTACACCGCGAGGAAACTTCCTCGCAAC |
| | TTTGTGGTAGATTACTATGAGACCAGCAGCCTCTGCTCCCA |
| | GCCAGCTGTGGTATTCCAAACCAAAAGAAGCAAGCAAGTCT |
| | GTGCTGATCCCAGTGAATCCTGGGTCCAGGAGTACGTGTAT |
| | GACCTGGAACTGAATTGATGAAAGCTT (SEQ ID NO: 19) |
| Translation of | MGSSHHHHHHHHQLFVKTLTGKTITLELEPSDTVENVKAKIQD |
| 3D3-MIP1β WT | KEGIPPDQQRLIFAGKQLEDGRTLSDYNIQKESTLHLVLRLRGG |
| | APMGSDPPTACCFSYTARKLPRNFVVDYYETSSLCSQPAVVFQ |
| | TKRSKQVCADPSESWVQEYVYDLELN* (SEQ ID NO: 20) |
| Ub-MIP1β WT | ATGGGCAGCAGCCATCATCATCATCATCACAGCAGCGGCCT |
| Features: | GGTGCCGCGCGGCAGCCATATGCAGATCTTTGTGAAGACCC |
| (1) His tag | TCACTGGCAAAACCATCACCCTTGAGGTCGAGCCCAGTGAC |
| (double- | ACCATTGAGAATGTCAAAGCCAAAATTCAAGACAAGGAGG |
| underlined); | GTATCCCACCTGACCAGCAGCGTCTGATATTTGCCGGCAAA |
| (2) Ubiquitin | CAGCTGGAGGATGGCCGCACTCTCTCAGACTACAACATCCA |
| encoding region | GAAAGAGTCCACCCTGCACCTGGTGTTGCGCCTCCGCGGTG |
| (underlined). | GAGCACCAATGGGCTCTGACCCTCCCACCGCCTGCTGCTTTT |
| | CTTACACCGCGAGGAAACTTCCTCGCAACTTTGTGGTAGAT |
| | TACTATGAGACCAGCAGCCTCTGCTCCCAGCCAGCTGTGGT |
| | ATTCCAAACCAAAAGAAGCAAGCAAGTCTGTGCTGATCCCA |
| | GTGAATCCTGGGTCCAGGAGTACGTGTATGACCTGGAACTG |
| | AATTGATGA (SEQ ID NO: 21) |
| Translation of | MGSSHHHHHHSSGLVPRGSHMQIFVKTLTGKTITLEVEPSDTIE |
| Ub-MIP1β WT | NVKAKIQDKEGIPPDQQRLIFAGKQLEDGRTLSDYNIQKESTLH |
| | LVLRLRGGAPMGSDPPTACCFSYTARKLPRNFVVDYYETSSLC |
| | SQPAVVFQTKRSKQVCADPSESWVQEYVYDLELN |
| | (SEQ ID NO: 22) |

To generate chemokines with native N-termini after Usp2-cc digestion, codon-optimized sequences were cloned into the pHUE vector using the SacII and HindIII restriction sites. Sequences for the codon-optimized and destabilizing ubiquitin variants were cloned using the Nde I and SacII restriction sites.

Fusion constructs were expressed using BL21(DE3)pLysS cells or TAP302 cells in Luria Broth at 30° C. or 37° C. Cultures were grown to an OD600 nm=0.6, in the presence of 200 μg/mL carbenicillin, and were induced using 1 mM IPTG for 3-4 hr. Cells were harvested via centrifugation at 5000×g for 15 min at 4° C. For test expressions, cell pellet weights were normalized by pelleting cells and resuspending them in lysis buffer (50 mM Tris 8.0, 300 mM NaCl, 5 mM MgSO4, 0.1% Tween-20) based on their relative OD600's. Cells were lysed through the addition of 10 μL of 1 mg/mL lysozyme followed by 2 rounds of freeze-thawing, and then spun down at 18,000×g for 10 min at room temperature. 5×SDS-loading buffer was added to the supernatant, while the pellets were resuspended in an identical volume of 1× loading buffer. 10 μL of pellet and supernatant from each sample fractions was run on 15% SDS-polyacrylamide gels and stained with Coomassie Blue. For large-scale purification, cell pellets were resuspended in 50 mM Tris 8.0, 300 mM NaCl, 10 mM imidazole. DNaseI, lysozyme (40 μg/mL) and EDTA-free protease inhibitor tablets (Roche, Indianapolis, Ind.) were added. Cells were lysed via sonication for 4×30 sec at 60% power, and the supernatant was clarified by centrifugation at 18,000×g for 15 min at 4° C. For soluble proteins, the supernatant was loaded onto a Ni-sepharose column (GE Healthcare, Piscataway, N.J.) or a Ni-NTA column (Qiagen, Valencia, Calif.), using an AKTA FPLC system (GE Healthcare). Peak fractions containing fusion proteins were eluted with 50 mM Tris 8.0, 300 mM NaCl, 500 mM imidazole, and dialyzed against 50 mM Tris 8.0, 300 mM NaCl prior to Usp2-cc cleavage and further purification.

### Refolding of Insoluble Ubiquitin-Chemokine Fusions

Cells were lysed and centrifuged as described herein. Pellets obtained after centrifugation were resuspended and washed twice with 20 mM Tris 8.0, 0.25% (w/v) Deoxycholate. Inclusion bodies were pelleted at 18,000×g for 15 min at 4° C. and then resuspended in 20 mM Tris 8.0, 6 M GuHCl using a Dounce homogenizer. The proteins were loaded onto a Ni-NTA column and eluted with 20 mM Tris 8.0, 6 M GuHCL 500 mM imidazole. All proteins were refolded prior to Usp2-cc cleavage, and refolding conditions were determined using the Fold-It screen (Hampton Research, Aliso Viejo, Calif.). Briefly, each fusion protein was rapidly diluted into each of sixteen refolding conditions. The refolded proteins were then dialyzed against 50 mM Tris 8.0, 300 mM NaCl, and conditions that produced no visible precipitation were used for larger scale purifications. The final refolding buffer conditions for each of the six chemokines tested in this study (CCL13, CCL27, CCL28, CXCL9-11). After refolding, chemokines were purified using Ni-affinity chromatography and dialyzed into cleavage buffer, as described herein. Refolding conditions are summarized in Table 5

TABLE 5

| Ubiquitin- | |
|---|---|
| chemokine | |
| fusions | Conditions |
| CCL13 | 55 mM Tris pH 8.0, 264 mM NaCl, 11 mM KCl, 0.055% |
| | PEG 3350, 1.1 mM EDTA, 1 mM DTT. |
| CCL27 | 55 mM Tris pH 8.0, 264 mM NaCl, 11 mM KCl, 550 mM |
| | GuHCl, 1.1 mM EDTA, 550 mM L-arginine-HCl, 1 mM |
| | reduced glutathione (GSH), 0.1 mM oxidized glutathione |
| | (GSSG). |
| CCL28 | 55 mM MES pH 6.5, 11 mM NaCl, 0.44 mM KCl, 550 mM |
| | GuHCl, 2.2 mM MgCl₂, 2.2 mM CaCl₂, 0.3 mM |
| | lauryl maltoside, 1 mM GSH, 0.1 mM GSSG. |
| CXCL9 | 55 mM MES pH 6.5, 11 mM NaCl, 0.44 mM KCl, 550 mM |
| | GuHCl, 2.2 mM MgCl₂, 2.2 mM CaCl₂, |
| | 440 mM sucrose, 0.3 mM lauryl maltoside, 1 mM |
| | GSH, 0.1 mM GSSG. |
| CXCL10 | 55 mM MES pH 6.5, 264 mM NaCl, 11 mM KCl, 1.1 mM |
| | EDTA, 440 mM sucrose, 1 mM GSH, 0.1 mM GSSG. |
| CXCL11 | 55 mM Tris pH 8.0, 264 mM NaCl, 11 mM KCl, 1.1 mM |
| | EDTA, 440 mM sucrose, 550 mM L-arginine-HCl, 1 mM |
| | DTT. |

### Expression and Purification of Usp2-cc

Usp2-cc was expressed and purified using a modified version of a previously published protocol. Briefly, pUsp2-cc was transformed into BL21(DE3) pLysS cells and grown in 2× Yeast Tryptone (2xYT) broth at 37° C. to an OD600 nm=0.6. Cultures were then induced with 1 mM IPTG and grown for a further 4 hrs. Cells were harvested via centrifugation at 5000×g for 15 min at 4° C. Pellets were resuspended in 50 mM Tris 8.0, 500 mM NaCl, 20 mM imidazole, 5% glycerol (v/v), 5 mM f3-mercaptoethanol, and sonicated for 4×30 sec @ 60% power. Cell debris was removed by centrifugation at 18,000×g for 15 min at 4° C. Supernatant was further clarified by filtering through a 0.22 μM filter (Millipore) before loading onto a Ni-Sepharose HP column (GE Healthcare) using an AKTA FPLC (GE Healthcare). Usp2-cc was eluted using 500 mM imidazole, and concentrated using 10 kDa-cutoff Amicon concentrators (Millipore, Billerica, Mass.). For final purification, Usp2-cc was loaded onto a Sephadex S75 size exclusion chromatography column (GE Healthcare), equilibrated with 50 mM TRIS, pH8.0, 300 mM NaCl, 1 mM DTT, 5% glycerol. Peak fractions containing Usp2-cc were concentrated as above, and glycerol was added to a final concentration of 25% (v/v). Purified enzyme was stored at −20° C.

### Cleavage of Ubiquitin-Chemokine Fusions by Usp2-cc, and Final Purification

Usp2-cc was incubated with dialyzed fusion protein at a molar ratio of 1:100 Usp2-cc: ubiquitin-chemokine Reactions were mixed and allowed to proceed at room temperature for typically 1-4 hrs. Most reactions went to completion within 2 hrs. To facilitate removal of His-tagged ubiquitin, Usp2-cc and any remaining fusion protein, an optional Ni-NTA column was added. Flow-through from this column containing chemokine was loaded onto a C18-reverse phase HPLC column (Grace, Deerfield, Ill.), pre-equilibrated with 0.1% Trifluoroacetic acid, 25% acetonitrile (AcN). Chemokines were eluted using a 25-90% gradient of AcN, lyophilized and stored at −80° C.

### Calcium Mobilization Assays

HEK293 cells expressing CCR1 were utilized. Calcium flux assays were performed using a FLIPR calcium 4 assay kit (Molecular Devices, Sunnyvale, Calif.), using 2×104 cells/well in a 384-well assay format. CCL7-dependent increases in cytosolic Ca²⁺ were measured using a FLIPR instrument (Molecular Devices).

### Results and Discussion:

Expression of ubiquitin-chemokine fusions: Eleven ubiquitin-chemokine fusions were transformed and tested for expression in BL21(DE3) pLysS cells. All of the chemokine fusions successfully expressed, although the total yield and the proportion of soluble protein varied, depending upon the chemokine Table 6 lists chemokines tested in this study, and their relative expression levels, as determined by gel analysis.

TABLE 6

| | | Soluble | Insoluble |
|---|---|---|---|
| | | expression at 4 hr | expression at 4 hr |
| Chemokine | Name | post-induction | post-induction |
| CCL2 | MCP-1 | ++ | ++++ |
| CCL3 | MIP-1α | +* | +++ |
| CCL7 | MCP-3 | +++ | + |
| CCL13 | MCP-4 | − | ++++ |
| CCL14 | HCC-1 (9-74) | ++ | ++ |
| CCL27 | CTACK | − | ++++ |
| CCL28 | MEC | − | ++++ |
| CXCL8 | IL-8 | ++++ | + |
| CXCL9 | MIG | − | ++ |
| CXCL10 | IP-10 | ++ | ++++ |
| CXCL11 | I-TAC | +* | ++++ |

*Small amounts of soluble protein were observed at 1 hr and 2 hr post-induction, but were not visible at 4 hr post-induction

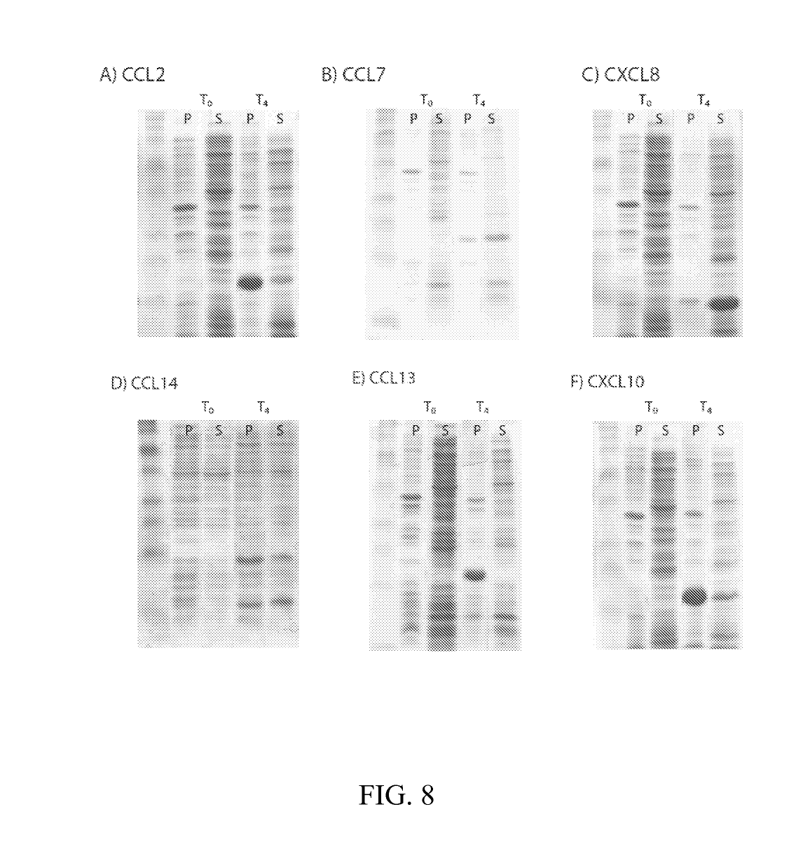

Five of the eleven fusions (CCL2, CCL7, CCL14, CXCL8 and CXCL10) showed soluble expression, although the estimated yield varied from ˜1 mg/L to >10 mg/L. The remaining fusions (CCL3, CCL13, CCL27, CCL28, CXCL9 and CXCL11) showed very little or no soluble expression, but they all showed good levels (>1 mg/L) of insoluble expression. This indicates that the presence of ubiquitin was unable to increase the solubility of otherwise insoluble chemokines, when expressed in this cell line. FIG. 8 shows a representative selection of the expression gels in which the amount of protein loaded in each lane was normalized to the cell density, and highlights differences in solubility and overall expression levels of the different chemokines tested.

As chemokines contain one or more disulfide bonds, it is not surprising that a number of them expressed insolubly in BL21(DE3) pLysS cells. To address this, the effect of using the disulfide-permissive TAP302 cells, and decreasing the expression temperature from 37° C. to 30° C., upon the yield of soluble protein was tested. The use of TAP302 cells to increase the yield of soluble His-tagged CCL2 has been reported. However, the combination of TAP302 cells and lower temperature did not enhance the solubility of the ubiquitin fusions (data not shown), so all further expressions were undertaken using BL21(DE3) pLysS cells.

Purification of chemokines: After successfully demonstrating that all of the chemokines that were tested as ubiquitin fusions, expressed at high levels. The fusions proteins were then tested to determine whether they could be successfully purified, and ubiquitin removed to generate active chemokine. For this study nine representative chemokines were selected (Table 8), three of which (CCL2, CCL7 and CCL14) were chosen to purify from soluble expression, while the other six (CCL13, CCL27, CCL28, CXCL9, CXCL10 and CXCL11) were predominantly insoluble, so were refolded from inclusion bodies before purification. Table 7 provides final yields and characterization of chemokines tested in this study.

TABLE 7

| | Insoluble | Yield of | Mass (Da) | | |
|---|---|---|---|---|---|
| | (I) or | purified | | Determined | |
| | soluble (S) | protein | Cal- | by | |
| Chemokine | expression | (mg/L) | culated | mass spec | Functional* |
| CCL2 | S | 1 | 8663.0 | 8662.8 | N.D. |
| CCL7 | S | 10 | 8952.4 | 8952.3 | Y (ref) |
| CCL13 | I | 3.5 | 8595.1 | 8595.0 | N.D. |
| CCL14 | S | 0.5 | 7796.8 | 7794 | Y |
| CCL27 | I | 30 | 10145.9 | 10145.0 | Y (ref) |
| CCL28 | I | 8 | 12068.0 | 12067.2 | N.D. |
| CXCL9 | I | 5 | 11748.8 | 11748.6 | N.D. |
| CXCL10 | I | 20 | 8642.3 | 8641.3 | N.D. |
| CXCL11 | I | 2.5 | 8303.0 | 8302.5 | N.D. |

*Chemokine function was tested by activity in a calcium flux assay, cell migration assay or chemokine receptor binding assay.
N.D. Not determined

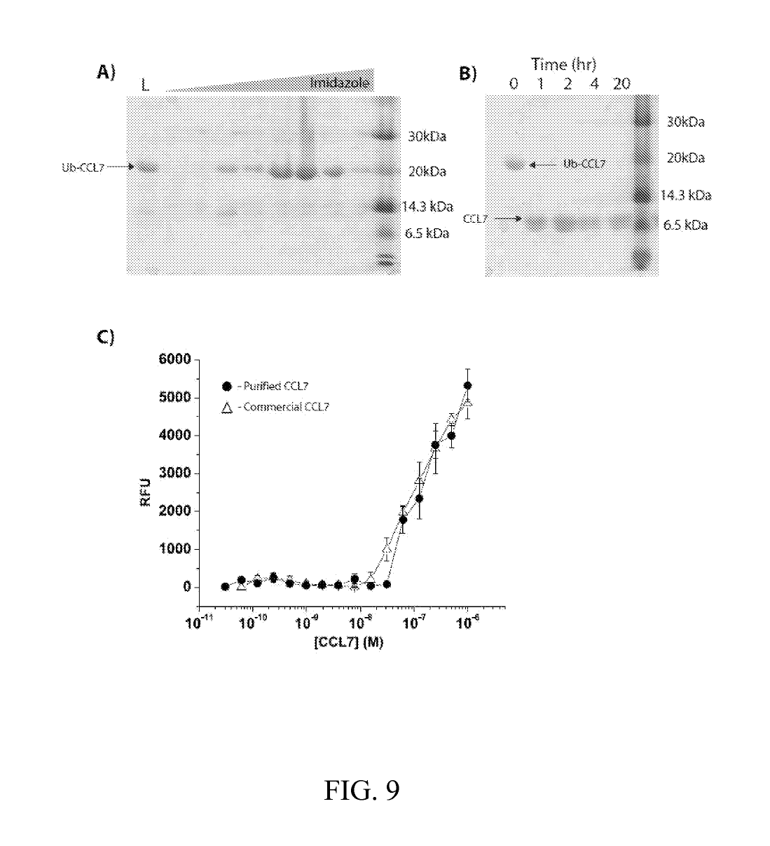

In order to purify these insoluble proteins, they were first solubilized in guanidine hydrochloride, which yielded up to 50 mg/L of fusion protein, and used a commercially available screen to help identify optimal refolding conditions for each protein. In all cases Ni-affinity chromatography as a first pass purification step was used, and this served to significantly enrich for the protein of interest. A representative gel from Ni-affinity chromatography of CCL7 is shown in FIG. 9A.

After partial purification, ubiquitin was removed from the fusions, using the catalytic core of the de-ubiquitylating enzyme Usp2-cc. FIG. 9B shows a typical result from the cleavage of the ubiquitin-CCL7 fusion. At a 100:1 molar ratio of fusion protein:Usp2-cc, the fusion was completely cleaved within 1 hr at room temperature. Cleavage rates and efficiencies were similar for the other chemokine fusions, although in some cases, a small amount of fusion protein was left uncleaved. This was most likely due to a small fraction of misfolded protein that was not recognized by the ubiquitinase. After further purification to remove Usp2-cc enzyme, uncleaved fusion protein and ubiquitin, all samples were tested by mass spectrometry to confirm that cleavage by Usp2-cc occurred at the correct site in the sequence. For each chemokine, only one species was observed by mass spectrometry, and this corresponded to the mass of the chemokine with the native N-terminus (Table 7), indicating that Usp2-cc cleaves specifically at the desired site.

The final yields of purified chemokine are shown in Table 7. In general, yields after purification and Usp2-cc cleavage tracked well with starting expression levels. For example, CCL7, CCL27 and CXCL10 all expressed very well, and could be purified to levels 10 mg/L, with CCL27 providing the highest final yield, at 30 mg/L. In contrast, CCL14 showed modest expression, and was purified with a final yield of 0.5 mg/L.

To confirm activity, selected chemokines were tested in chemokine binding, calcium flux and/or chemotaxis assays (FIG. 9C). To compare activity of CCL7 expressed as an ubiquitin fusion to commercially available CCL7 directly, the effects of CCL7 from the two sources upon calcium mobilization in CCR1-expressing HEK293 cells was tested (FIG. 9C). Both proteins showed identical activity in this assay, indicating that CCL7 expressed as an ub-fusion behaves very similarly to that obtained by other methods.

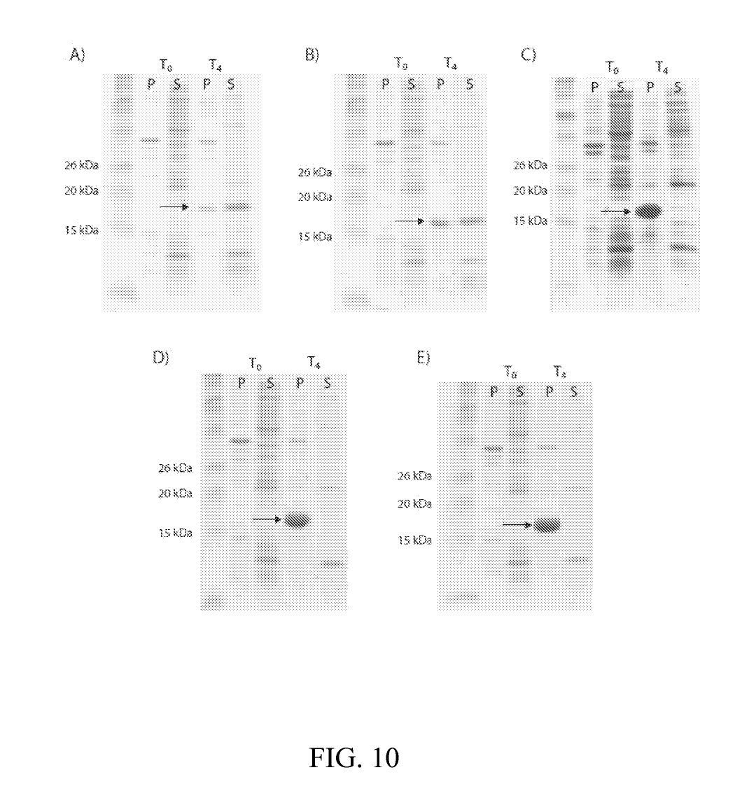

Optimization and redesign of ubiquitin: To explore whether it would be possible to increase chemokine yields, a number of different forms of ubiquitin was tested. Initially the wildtype ubiquitin DNA sequence was replaced with one that had been optimized by mutating the rare arginine codons. When fused to CCL7, this new optimized sequence did appear to slightly increase the overall yield, though primarily through increasing the expression of the insoluble fraction (FIG. 10A vs FIG. 10B). Similar results were also observed using optimized fusions to CCL3 and CCL13 (data not shown). In some cases, as with CCL28 and CXCL11, no appreciable increase in expression could was seen. It is unclear why the benefit from codon optimization varied among the fusions.

Since the use of a codon optimized sequence tended to increase the insoluble expression of the fusions, a variety of destabilized ubiqutin proteins to try and drive the expression into inclusion bodies was used. A number of computationally core-repacked mutants of ubiquitin were generated and characterized. Three of these mutants, 3D3, 2D6 and *R6 were selected, to cover a range of destabilizing mutations. To test the effect of these mutants to promote inclusion body expression CCL7 was used, which normally expresses solubly. In all three cases, there was a significant increase in the overall expression level and the resulting destabilized ubiquitin-fusion expressed insolubly (FIG. 10A, versus FIG. 10C, FIG. 10D, and FIG. 10E). It should be noted, that some soluble expression was observed at intermediate time points, but by the final harvest point all of the fusion was seen in the inclusion body. Similar results were seen for other chemokines.

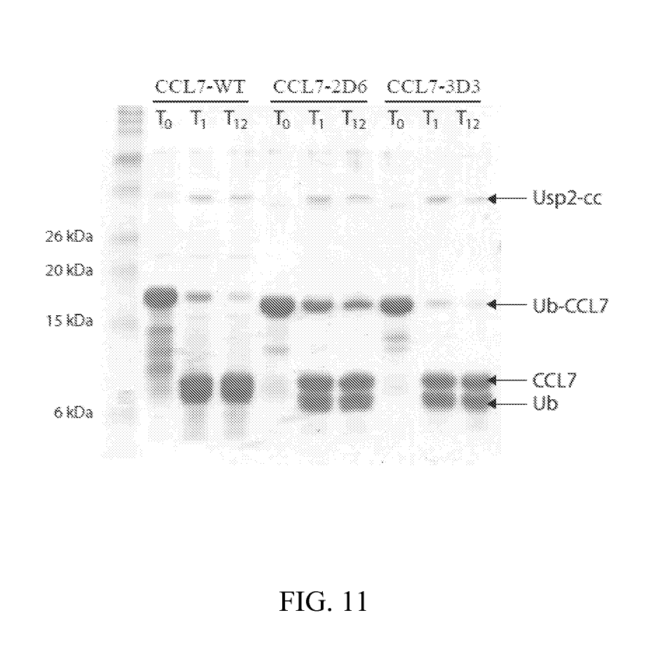

One concern with using the core-repacked ubiquitin mutants was whether these variants could be efficiently recognized and cleaved using Usp2-cc. To test this, refolding and cleavage studies using CCL7 as a test case were carried out (FIG. 11). 3D3 ubiquitin (3D3-CCL7) was recognized and cleaved with efficiency similar to that observed for wildtype ubiquitin (WT-CCL7). In contrast, the 2D6 fusion (2D6-CCL7) was only partially cleavable, with large amounts of fusion remaining uncleaved after 12 hr. Based on the published CD spectra for the two mutant proteins, the cleavage efficiency could be related to how close to wildtype the spectra are.

Expressing chemokines as ubiquitin fusion proteins, and subsequently cleaving using Usp2-cc, it is possible to routinely express and purify milligram quantities of pure, active chemokines. Based on our experience with both major families of chemokines, this system is generally applicable to the production of most, if not all chemokines Finally, by driving the expression of insoluble protein, destabilizing mutants of ubiquitin such as ub-3D3 show promise in their ability to improve the yield of otherwise low expressing chemokines

### Example 9

### A Rapid and Efficient Method to Obtain Modified Chemokines

Chemokines include a large family of signaling molecules that are produced by a variety of cells in response to signals associated with host defense and wound repair. They play a pivotal role in the immune system, controlling migration and activation of leukocytes, by binding to and signaling through seven transmembrane G-protein coupled receptors (GPCRs) on the surface of leukocytes [1, 2]. However, chemokines and their receptors are often likened to double-edged swords because they are also associated with a number of pathologies and are essential cofactors during viral entry of HIV into host cells [3-5].

Studies of chemokine:receptor binding in intact cells or membrane fragments have been traditionally conducted using ¹²⁵I-radiolabeled chemokines. However, this method has a number of drawbacks, including changes in receptor binding affinity caused by iodination of the chemokine, and the necessity of custom labeling if a particular chemokine of interest is not commercially available. Also, the assays are time-consuming and require multiple wash steps to remove unbound chemokine. More recently, homogeneous assays such as the Scintillation Proximity Assay (SPA) (PerkinElmer) have become popular, particularly in the pharmaceutical industry, as they can be easily miniaturized and require no wash steps. However, they still use radiolabeled chemokines that may need to be custom-labeled, and require safe handling procedures and hazardous waste disposal.

It is possible to express, purify and solubilize GPCRs, including chemokine receptors at sufficient levels for structural and biophysical studies [6-11]. Progress in this area recently culminated in the first high resolution crystal structures of a chemokine receptor; those of CXCR4 in complex with small molecule and peptide antagonists [7]. Nevertheless, there are few reports describing chemokine binding to their receptors in solution and due to the lack of alternative approaches, radiolabeled ligands (e.g. ¹²⁵I-chemokine and ³⁵S-GTP-γS) have been used for this purpose too. Expression and purification of non-radiolabeled chemokines has been described previously [12-14], though sporadically, and for only one or two ligands. Chemical synthesis of fluorescently labeled chemokines is also possible [15], but is expensive, making the use of these ligands impractical for large-scale studies.

Described herein are methods to systematically express and purify milligram quantities of four different functional, derivatized chemokines for use in a range of assays. To date, fluorescent and/or biotinylated versions of CCL7, CCL14, CCL3 and CXCL8 have been obtained; this method is also valid for many other chemokines. The applications tested in this study include non-traditional radioligand assays, in which biotinylated chemokines are used in conjunction with ¹²⁵I-labeled streptavidin, circumventing the need for radiolabeled chemokines and problems associated with reduced binding affinity of the radiolabeled probe. In addition, described herein is the use of biotinylated chemokines for use as ligand affinity columns, and the production of fluorescently labeled chemokines for solution-based fluorescence anisotropy-based binding assays. The ability to readily produce these chemokine reagents should also aid in many other types of studies including fluorescence resonance energy transfer-based studies, drug discovery efforts, and array based screening of interacting partners such as chemokine:glycosaminoglycan interactions [16].

### Materials and Methods

Fluorescein-5-maleimide and Alexa Fluor 647 C2-maleimide were obtained from Invitrogen (Carlsbad, Calif.). E. coli codon optimized variants of full-length CCL3, CCL7, and CXCL8, along with the functional form of CCL14 containing residues 9-74 [17] were purchased from Genscript (Piscataway, N.J.). The pHUE vector containing an N-terminal His tag and ubiquitin (ub), along with pET15b containing the catalytic core of the deubiquitylating enzyme Usp2 (Usp2-cc) were obtained from JCSMR, The Australian National University, ACT0200, Can berra, Australia. A variant of ubiquitin in which three destabilizing mutations were introduced (I3L, V17L, I23V) [18] was cloned into pET27 (EMD Chemicals, Gibbstown, N.J.) in-house to obtain pHUE-3D3. ¹²⁵I-labeled CCL3 and streptavidin were purchased from Perkin Elmer (Waltham, Mass.), and anti-CCR1 antibody was obtained from Thermo Fisher Scientific (Rockford, Ill.).

Cloning and Expression of Chemokines as pHUE- and pHUE3D3-Chemokine Fusions

Chemokines were cloned onto the 3′ end of ubiquitin in pHUE [19] (CCL7, CCL14) or ubiquitin-3D3 in pHUE-3D3 (CCL3, CXCL8) using SacII and HindIII restriction sites at the chemokine N and C-termini respectively. For derivatization, DNA coding for a cysteine residue or an Avitag sequence (KLGSGLNDIFEAQKIEWHE; SEQ ID NO:23) was also added to the C-terminus of the chemokine. After verification by sequencing, pHUE or pHUE3D3-based constructs were transformed into BL21(DE3)pLysS cells (Novagen, Madison, Wis.). For pHUE constructs, cells were grown in Luria Broth in the presence of carbenicillin (200 μg/mL) and chloramphenicol (34 μg/mL) at 30° C. For pHUE3D3 constructs, cells were grown in Luria Broth in the presence of kanamycin (40 μg/mL) and chloramphenicol (34 μg/mL) at 37° C. In all cases, expression of the ubiquitin-chemokine fusions was induced using 0.5 mM IPTG at OD₆₀₀ₙₘ ˜0.6. After induction, cells were grown for 4 hours before harvesting.

### Chemokine Purification and Derivatization

Cell pellets containing ubiquitin-chemokine fusions were resuspended in buffer A (50 mM HEPES, pH 7.4, 0.5 M NaCl, 40 mM imidazole, protease inhibitor tablets (no EDTA) (Roche, Indianapolis, Ind.)). 5 mM MgCl₂, DNase I (Roche) and 0.1% Tween-20 (Sigma, St. Louis, Mo.) were added and cells were disrupted using an Emulsiflex (Avestin, Ottawa, Canada) (2 runs, 12,000 psi). For soluble chemokines expressed using pHUE, insoluble material was removed by centrifugation (48,000 g, 45 minutes, 4° C.) and the supernatant was loaded onto a Ni-sepharose column (GE Life Sciences, Piscataway, N.J.). Ub-chemokine fusion proteins were eluted using a linear gradient from 40 mM to 500 mM imidazole. CCL3 expressed predominantly insolubly as a pHUE3D3 fusion, so was purified and refolded from inclusion bodies before being loading onto a Ni-affinity column as described above.

Inclusion body preparation and refolding of CCL3 was undertaken according to a modified version of Zabel et al. [20]. Briefly, cell pellets were detergent-solubilized by three rounds of homogenization and centrifugation (40,000 g, 45 mins) in the presence of 0.25% (w/v) sodium deoxycholate. The insoluble inclusion body pellet was solubilized in a denaturing buffer (6 M guanidine HCl, 0.1 M sodium phosphate, 10 mM Tris, pH8.0) and loaded onto a nickel-nitrilotriacetic acid (Ni-NTA) column (Qiagen, Valencia, Calif.). CCL3 was eluted by decreasing the pH to 4, and refolded by rapid dilution into refolding buffer (55 mM MES, pH 6.5, 264 mM NaCl, 11 mM KCl, 0.055% (w/v) PEG3350, 1.1 mM EDTA, 550 mM L-arginine, 0.3 mM n-dodecyl maltoside, 1 mM reduced L-glutathione, 0.1 mM oxidized L-glutathione), at a final CCL3 concentration of 0.1 mg/mL. After stirring overnight at 4° C., refolded CCL3 was dialyzed into buffer A before loading onto a Ni-affinity column. After Ni-affinity chromatography, ubiquitin fusion proteins were either directly cleaved using the Usp2-cc, or further purified by loading onto a C18 reversed-phase liquid chromatography column (Grace Vydac, Deerfield, Ill.) in 0.1% trifluoroacetic acid (TFA)/25% acetonitrile and eluted using an increasing gradient of acetonitrile. In the latter case, chemokines were lyophilized and stored as fusion proteins.

For deubiquitylation by Usp2-cc, samples were resuspended to approximately 1 mg/mL in a buffer containing 20 mM Tris, pH 8.0, 0.2 M NaCl. Usp2-cc, prepared as described in Catanzariti et al. [19], was added at a molar ratio of 50:1 chemokine:Usp2-cc, and samples were placed at room temperature for 2 hours before loading onto a Ni-NTA column (Qiagen) in 50 mM Tris, pH 7.5, 40 mM imidazole, 500 mM NaCl. Ni-NTA was used at this stage instead of Ni-sepharose, due to the tendency of untagged chemokines to bind to Ni-sepharose resin (data not shown). Column flow-through samples containing chemokine were quickly pooled and derivatized by addition of fluorophore or BirA enzyme as described below. For fluorescent labeling, chemokines containing a C-terminal cysteine were mixed with a 1:5 molar ratio of chemokine to fluorescein-5-maleimide or Alexa Fluor 647, and incubated in the dark at 4° C. overnight. Biotinylation of Avitagged chemokines was undertaken using the BirA enzyme, prepared as previously described [21]. After derivatization, labeled chemokines were further purified using a C18 reversed-phase liquid chromatography column as described above. This step efficiently removed unbound small molecules such as fluorophore and biotin, along with BirA and any remaining unbiotinylated chemokine. After purification, samples were flash-frozen and lyophilized. The identity and purity of all samples was verified using electrospray mass spectrometry.

### Cell Culture and Calcium Mobilization Assays

HEK293 cells expressing CCR1 were cultured as previously described [8]. Calcium flux activity assays were performed using a FLIPR Calcium 4 assay kit (Molecular Devices), using 1.3×105 cells per well in a 96-well assay format. Chemokine-dependent increases in cytosolic Ca2+ were measured using a FlexStation 3 microplate reader (Molecular Devices).

### Construction of Chemokine Affinity Columns

For each column, 200 μL slurry of high capacity streptavidin agarose resin (Thermo Scientific) was equilibrated in 50 mM Tris, pH 8.5, 150 mM NaCl. Resin was mixed with 1 mg biotinylated CCL14 or buffer alone in a total volume of 1 mL. After 20 minute incubation at 20° C., columns were washed with 40 column volumes of equilibration buffer followed by binding buffer (50 mM Tris pH 8.5, 150 mM NaCl, 10% glycerol, 0.1% (w/v) dodecyl maltoside and 0.01% (w/v) cholesterol hemisuccinate). Detergent-solubilized CCR1, prepared as previously described [8], was loaded onto the CCL14 and control columns by gravity flow, and the column was washed three times with 1 mL binding buffer. CCR1 was eluted by washing twice with 1 mL binding buffer supplemented with 1 M NaCl.

### Radioligand Binding Assays

Scintillation proximity assay (SPA) binding assays (GE Healthcare, Piscataway, N.J.) were conducted essentially as previously described [8, 22, 23]. HEK293 cells stably expressing CCR1 were used for these assays, and untransfected HEK293 cells were used as a control. Assays were undertaken in a 96-well plate format and each data point was assayed in triplicate on a Microbeta plate counter (Perkin Elmer) for 1 min per well. Each well contained 20,000 cells, 0.2 mg WGA-PVT-SPA beads, increasing concentrations of unlabeled competitor chemokine, and either 50 μM ¹²⁵I-CCL3 or 100 μM ¹²⁵I-Streptavidin +400 pM CCL3-Biotin, in a total volume of 100 μL. IC₅₀ values were obtained by nonlinear regression curve fitting using Prism software (GraphPad, La Jolla, Calif.).

### Results and Discussion

### Purification of Chemokines

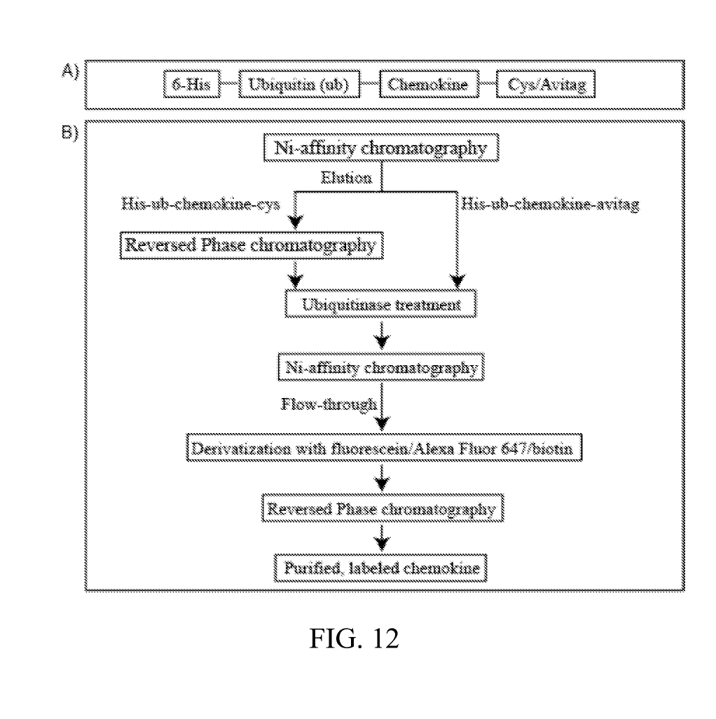

E. coli codon-optimized variants of four chemokines (CCL3, CCL7, CCL14, and CXCL8) were cloned into pHUE and pHUE3D3 vectors to produce a fusion protein containing an N-terminal 6-His tag, ubiquitin and the chemokine of interest. Depending upon the application, the chemokine was either native, or modified at the C-terminus by addition of an extra cysteine residue or an Avitag sequence, to permit labeling (FIG. 12A). The N-terminal ubiquitin fusion system was developed, as cleavage with Usp2-cc results in the native sequence. This is important because the exact nature of the N-terminal residues of chemokines affects their activity. In contrast, chemokines are generally tolerant to modifications at their C-termini, so this region was chosen for the addition of non-native residues for labeling. Two different versions of ubiquitin were used in this study: wildtype ubiquitin (pHUE vector) was used for expression of CCL7, CCL14 and CXCL8, as these express solubly at high levels and ubiquitin promotes soluble expression. However, a core-repacked mutant of ubiquitin (Ub3D3 in the pHUE-3D3 vector) was used for the expression of CCL3, in order to increase the overall yield of this protein by enhancing its expression in inclusion bodies.

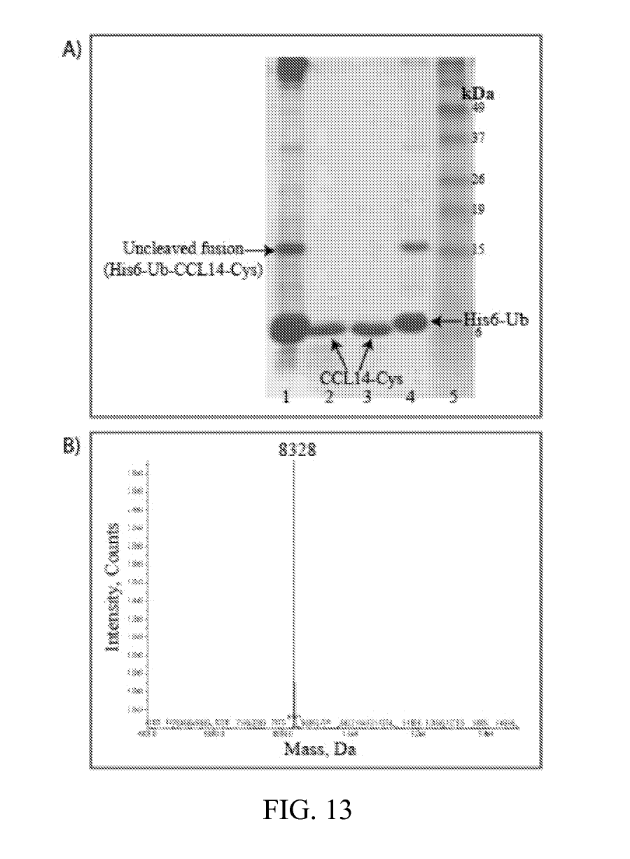

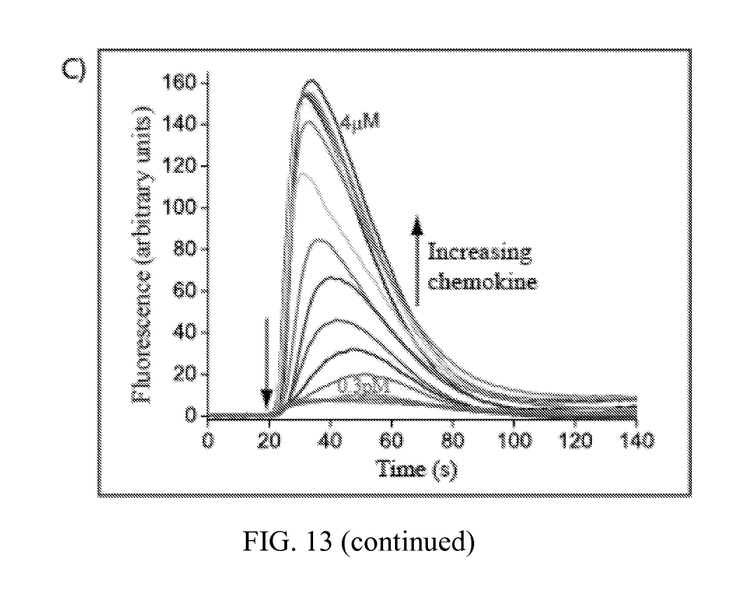

An overview of the purification scheme is described in FIG. 12B, and representative gels and mass spectrometry data for CCL14 are shown in FIG. 13A/B as an example. For the Avi-tagged chemokine constructs, 6-His-ubiquitin-chemokine-Avitag fusions were purified by Ni-affinity chromatography, the chemokines were cleaved from 6-His-ubiquitin using Usp2-cc, derivatized as required and loaded onto Ni-NTA resin prior to a final reverse phase chromatography and lyophilization. As ubiquitin elutes from a C18 reverse phase chromatography column at a similar acetonitrile concentration as many chemokines, the addition of a Ni-NTA column served to remove His-tagged ubiquitin, and increase the purity of the final product. This procedure was modified slightly for chemokines containing C-terminal cysteine residues such that an additional Reversed Phase HPLC column was inserted into the method prior to cleavage and derivatization. This optional additional step allowed chemokines to be stored in an underivatized state with minimal aggregation, as described herein. As shown in FIG. 13 for CCL14, cleavage of ubiquitin-chemokine fusion is >90% efficient (FIG. 13A, lane 1), and the column efficiently separates chemokine that flows through the column (FIG. 13A, lanes 2 and 3) from His-tagged ubiquitin that efficiently binds to the resin (FIG. 13A, lane 4). After purification, mass spectrometry confirmed the correct mass of the purified, derivatized proteins (FIG. 13B). Table 8 provides a summary of masses of labeled chemokines, and final yields after derivatization

TABLE 8

| | Mass of derivatized protein by | | | Yield of |
|---|---|---|---|---|
| | electrospray mass spectrometry (Da) | | | derivatized |
| | | +Alexa | | chemokine |
| Chemokine | + Fluorescein | Fluor 647 | +Biotin | (mg/L) |
| CCL7/MCP-3 | 9482.2 | 10038.3 | nd | 2.5 |
| | (Expected: 9482.3) | | | |
| CCL14/HCC-1 | 8328 | 8881 | 10220 | 0.5 |
| | (Expected 8326.9) | | | |
| CCL3/MIP-1α | nd | nd | 10138 | 4.0 |
| CXCL8/IL-8 | nd | nd | 10575.5 | 5.0 |

### Fluorescent Labeling of CCL7 and CCL14

Fluorescently labeled chemokines are attractive as they can be used to easily determine dissociation constants for the binding of chemokines to solubilized chemokine receptors [8]. In order to obtain fluorescently labeled chemokines, the proteins were expressed with a non-native C-terminal cysteine, and maleimide chemistry was used to couple fluorescent labels at this position after purification.

During preliminary purifications of CCL7-cys, cleavage of ubiquitin early in the purification and store the protein as lyophilized underivatized protein was attempted, as was customary for wildtype CCL7. However, after cleavage and storage the efficiency of derivatization was very low indicating that the reactive cysteine was not inaccessible. Electrospray mass spectrometry confirmed that chemokine dimers had formed, as the major mass observed was 18108.5 (expected mass of dimer=18109.0). This dimer was presumably facilitated by the formation of an additional time-dependent disulfide bond between the non-native C-terminal cysteine residues.

Chemokines contain multiple disulfide bonds (CCL3, CCL7, CCL14 and CXCL8 each contain two) and attempts to selectively reduce the non-native disulfide bond with reasonable efficiency failed. However, in small-scale purification studies the CCL7 fusion protein was monomeric until the ubiquitin was cleaved, based on SDS-PAGE gels run under non-reducing conditions. Therefore, purification of the chemokine as a fusion was carried out, and remove ubiquitin as a last step, just prior to derivatization. This method was successful for both chemokines tested, and the yields of purified CCL7 and CCL14 prior to derivatization were approximately 5 mg/L and 1 mg/L cells respectively. How ubiquitin is able to prevent dimerization of chemokines is unclear without the existence of a high-resolution structure, but is likely due to steric hindrance. The recovery of pure, labeled CCL7 and CCL14 was 2.5 mg/L and 0.5 mg/L respectively (Table 8).

Fluorescently tagged chemokines displayed activity indistinguishable from native chemokines on cell membranes [8]. FIG. 13C shows representative calcium flux data generated by exposing HEK293s cells stably expressing CCR1 to a dilution series of CCL14-fluorescein. The EC₅₀ for the dose response curve was 2 nM, similar to that (2.8 nM) previously published for unlabeled CCL14 [17]. Fluorescently labeled chemokines may be used in fluorescence polarization studies to obtain dissociation constants for chemokine binding to solubilized chemokine receptors [8].

### Biotinylation of CCL3, CCL14 and CXCL8

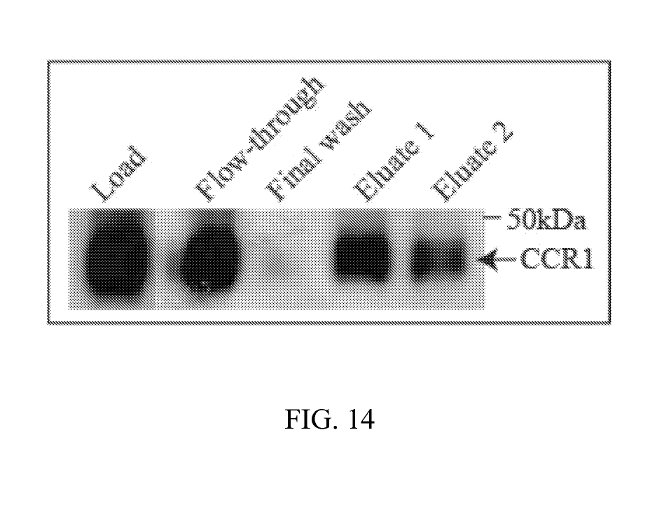

The addition of an Avitag, and biotinylation with the BirA enzyme is a well characterized method for producing protein that is biotinylated at a single, specific lysine residue [21]. Biotin has been used as a molecular handle in numerous published studies, but the ability to biotinylate chemokines is especially attractive because chemokine ligand affinity columns can be used to separate functional chemokine receptors from non-functional chemokine receptors during receptor purification. Three chemokines (CCL3, CCL14 and CXCL8) were tested in this study, and all three could be purified and biotinylated with an efficiency of >95%. In all three cases, the final yield of biotinylated chemokine was at least 0.5 mg/L, and masses were as expected according to electrospray mass spectrometry (Table 8). To demonstrate the utility of this approach, CCR1 solubilized in detergent micelles [8] was loaded onto a column containing biotinylated CCL14 immobilized on streptavidin-agarose resin. FIG. 14 shows a representative anti-CCR1 western blot of column samples from this experiment. The CCR1 sample likely contains a mixture of functional and non-functional CCR1, as illustrated by the presence of CCR1 in both the flow-through and the elution fractions of the chemokine column. A control column without immobilized CCL14 did not bind CCR1.

Another useful application of biotinylated chemokines is in non-conventional radioligand binding assays. Radioligand assays are accepted as the gold-standard for determining binding affinities between chemokines and their receptors, and as such are desired for chemokine receptor studies. The drawback of this approach is that although many common chemokines can be purchased as ¹²⁵I versions, some chemokines need to be custom labeled, and it is generally accepted that iodination can affect receptor binding affinities. However, it is possible to purchase ¹²⁵I-labeled streptavidin, and to use it in conjunction with biotinylated chemokine as a surrogate for labeled chemokine in binding assays.

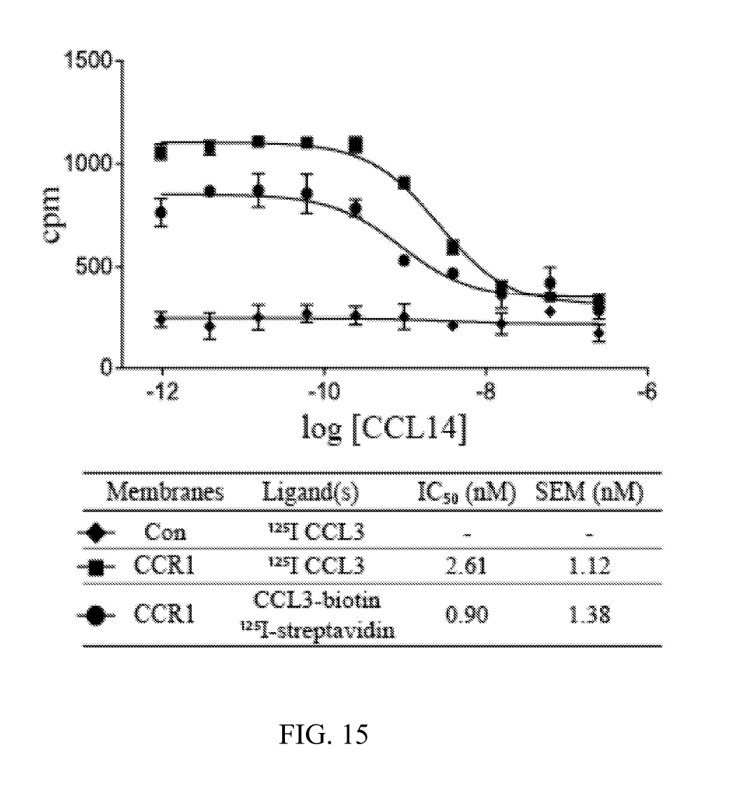

In order to test the use of ¹²⁵I-streptavidin as a surrogate radiolabel in a chemokine SPA assay, competition assays using membranes containing the chemokine receptor CCR1 were performed. CCL3 was used for these assays because ¹²⁵I-CCL3 is commercially available, allowing for a direct comparison. After immobilization of membranes to SPA beads, either radiolabeled chemokine, or a combination of biotinylated chemokine and ¹²⁵I-labeled streptavidin was added. FIG. 15 shows a representative SPA assay for CCR1, and demonstrates that unlabeled CCL14 competes with ¹²⁵I-CCL3 or a combination of CCL3-biotin/¹²⁵I-streptavidin for binding to HEK293 membranes containing CCR1 with a similar affinity (IC₅₀=2.6 nM versus 0.9 nM for displacement of ¹²⁵I-CCL3 or CCL3-biotin/¹²⁵I-streptavidin respectively). In contrast, no binding of CCL3 or competition with CCL14 was observed when CCR1-expressing membranes were replaced with untransfected HEK293 membranes. In addition, the ¹²⁵I-Streptavidin signal was CCL3-biotin dependent and while the highest signal was observed with a 4:1 ratio of chemokine to streptavidin, avidity effects due to the tetrameric state of streptavidin were not observed. These data indicate that the assay is a valid surrogate for traditional radioligand assays, and opens the door to radioligand assays using any chemokine, while only purchasing a single radiolabeled species (¹²⁵I-streptavidin). These assays may be further improved with appropriately engineered versions of monomeric streptavidin [24].

Accordingly, it is possible to efficiently and inexpensively produce milligram quantities of labeled chemokines that can be used in many cell-based, biochemical and biophysical assays. These assays include fluorescence-based binding assays, radioligand binding assays, and the generation of chemokine affinity columns. Easily separating functional chemokine receptors from non-functional counterparts, and conducting binding studies in solution enables the identification of detergents/lipids that retain receptors in a chemokine-binding competent state [8].

### Example 10

### Ubiquitin-Chemokine Fusion Constructs

DNA constructs comprising the 3D3 ubiquitin encoding nucleic acid fused to nucleic acids encoding various chemokines were generated and are summarized in Table 9.

TABLE 9

| Vector Name | Ubiquitin | Chemokine | Species |
|---|---|---|---|
| pCEV-33 | 3D3 | CXCL11 | human |
| pCEV-34 | 3D3 | CCL13 | human |
| pCEV-35 | 3D3 | CCL28 | human |
| pCEV-36 | 3D3 | CCL3 | human |
| pCEV-37 | 3D3 | CCL2 | human |
| pCEV-38 | 3D3 | CXCL8 | human |
| pCEV-39 | 3D3 | CXCL9 | human |
| pCEV-40 | 3D3 | CXCL10 | human |
| pCEV-50 | 3D3 | CCL2 | murine |
| pCEV-51 | 3D3 | CCL19 | murine |
| pCEV-52 | 3D3 | CCL20 | murine |
| pCEV-53 | 3D3 | CCL21 | murine |

### Example 11

### 3D3 Ubiquitin Moiety Protects Fusion Polypeptides from Degradation

Fusion polypeptides comprising either: (1) a wildtype ubiquitin moiety and CCL3/MIP-1α (ub-MIP-1α); or (2) a 3D3 ubiquitin moiety and CCL3/MIP-1α (3D3-MIP-1α), were expressed in E. coli. Sequences are shown in Table 10.

TABLE 10

| Ubiquitin | Sequence |
|---|---|
| Wildtype with His | MGSSHHHHHHSSGLVPRGSHMQIFVKTLTGKTITLEVEPSDTIENVKA |
| tag | KIQDKEGIPPDQQRLIFAGKQLEDGRTLSDYNIQKESTLHLVLRLRGG |
| (SEQ ID NO: 24) | |
| 3D3 with His tag | MGSSHHHHHHHHQLFVKTLTGKTITLELEPSDTVENVKAKIQDKEGIP |
| (SEQ ID NO: 25) | PDQQRLIFAGKQLEDGRTLSDYNIQKESTLHLVLRLRGG |
| 3D3 without His | QLFVKTLTGKTITLELEPSDTVENVKAKIQDKEGIPPDQQRLIFAGKQ |
| tag | LEDGRTLSDYNIQKESTLHLVLRLRGG |
| (SEQ ID NO: 26) | |

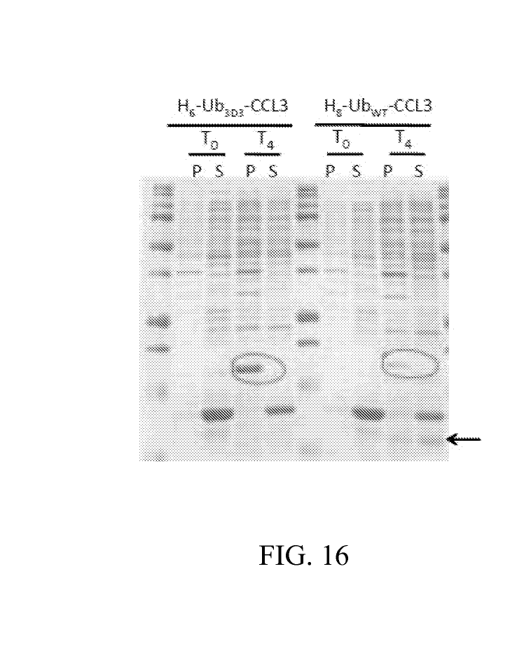

FIG. 16 shows protein expression of a 3D3-MIP-1α polypeptide (left) or ub-MIP-1α polypeptide (right). Expression fractions were taken at time 0 and 4 hours post induction and divided into pellet/insoluble (P) and supernatant/soluble (S) fractions. Full length fusion protein is circled in red and degradation products are indicated with arrows.

Each polypeptide was expressed in an insoluble form. Cleavage products of ub-MIP-1α were observed in expression fractions. In contrast, no cleavage products of 3D3-MIP-1α were observed in expression fractions. Thus, the 3D3 ubiquitin moiety protects the linked fusion polypeptide from degradation. This protection increases the yield of a fusion polypeptide linked to a 3D3 ubiquitin moiety, compared to the yield of a fusion polypeptide linked to a wild type ubiquitin moiety.

### Example 12

### 3D3 Ubiquitin Moiety Increases Expression into Inclusion Bodies

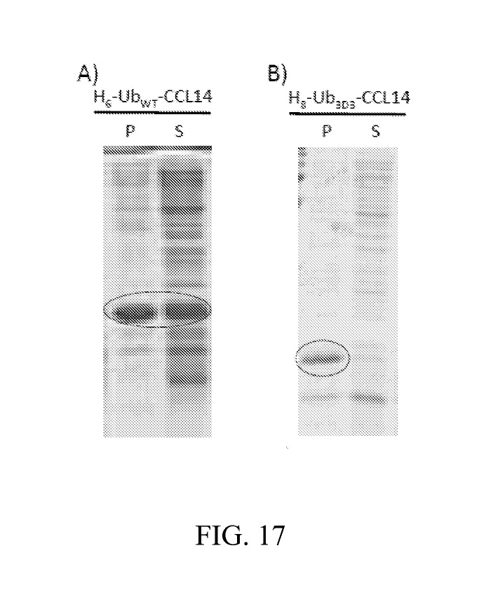

Fusion polypeptides comprising either (1) a wildtype ubiquitin moiety and CCL14 (ub-CCL14); or (2) a 3D3 ubiquitin moiety and CCL14 (3D3-CCL14), were expressed in E. coli. FIG. 17 shows protein expression of ub-CCL14 (A) and 3D3-CCL14 (B). Expression fractions were at 4 hours post induction and divided into pellet/insoluble (P) and supernatant/soluble (S) fractions. Full length fusion protein is circled in red.

The ub-CCL14 fusion polypeptide was expressed in both the soluble (supernatant) and insoluble (pellet/inclusion body) fractions. In contrast, the 3D3-CCL14 fusion polypeptide was expressed entirely in insoluble (pellet/inclusion body) fractions. This demonstrates the utility of the 3D3 system and its destabilizing effect on chemokine expression to enhance protein yields.

### Example 13

### 3D3 Ubiquitin Moiety is a Useful Expression System when the Quality of the Soluble Fusion Protein is not Optimal

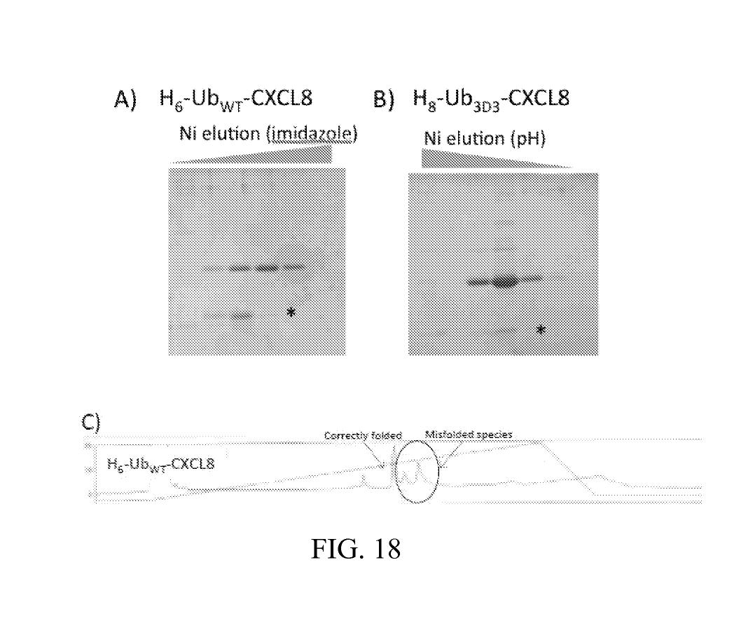

Fusion polypeptides comprising either (1) a wildtype ubiquitin moiety and CXCL8 (ub-CXCL8); or (2) a 3D3 ubiquitin moiety and CXCL8 (3D3-CXCL8), were expressed in E. coli. FIG. 18 shows protein purification of ub-CXCL8 (A) and 3D3-CXCL8 (B). The ub-CXCL8 fusion polypeptide was expressed mostly in a soluble fraction, and was purified over a Ni-NTA column and eluted with increased imidazole. The 3D3-CXCL8 fusion polypeptide was expressed mostly in insoluble inclusion bodies, and was purified over a Ni-NTA column and eluted with decreased pH. Although the ub-CXCL8 fusion polypeptide appeared as a uniform species by SDS-PAGE (A), an HPLC chromatogram of this material revealed that there were multiple species containing both folded and misfolded proteins (C). In particular, while the ub-CXCL8 fusion polypeptide was expressed in a soluble fraction, HPLC analysis indicated that about 30-40% of the expressed ub-CXCL8 fusion polypeptide was misfolded. The misfolded fusion polypeptide was prone to precipitation during dialysis, resulting in a further decreased protein yield. Here, the use of the 3D3 ubiquitin moiety provided a good alternative expression and purification strategy. Expression of the 3D3-CXCL8 fusion polypeptide was greater than expression of the ub-CXCL8 fusion polypeptide, with >90% of the 3D3-CXCL8 fusion polypeptide expressed into inclusion bodies (data not shown). Both the ub-CXCL8 and the 3D3-CXCL8 fusion polypeptides produced ubiquitin cleavage products (denoted by *), however, the ub-CXCL8 fusion polypeptide exhibited a greater level of cleavage.

Table 11 summarizes the results of Examples 11-13. In 3 out of 4 cases shown in Table 11, 3D3 fusion polypeptides had at least a 2-fold increase in yield (mg/L) compared to the wildtype ubiquitin fusion polypeptides. These examples demonstrate the advantages of using 3D3-ubiquitin moieties to express fusion polypeptides where the polypeptide is: (1) highly prone to degradation (e.g. CCL3); (2) partitioned into both soluble and insoluble fractions (e.g. CCL14); or (3) is misfolded in the soluble fraction (e.g. CXCL8). Use of the 3D3 system overcomes these issues, in part, by increasing expression of the fusion polypeptide into inclusion bodies.

TABLE 11

| | Distribution of fusion | | Yield of | Yield of fusion | |
|---|---|---|---|---|---|
| | polypeptide in | | refolded | polypeptide | |
| Fusion | pellet/supernatant fractions | | fusion | (mg/L) | |
| partner | Ub_{WT} | Ub_{3D3} | polypeptide | Ub_{WT} | Ub_{3D3} |
| CCL3 | 100% pellet, | 100% pellet, | ~90% | 3 | 20 |
| | degradation | no degradation | | | |
| | products | products | | | |
| CCL2 | 85% pellet | No change | ~100% | 10 | 10 |
| CXCL8 | 95% supe* | 90% pellet | ~100% | 6 | 15 |
| | (part | | | | |
| | misfolded) | | | | |
| CCL14 | 50% pellet | 100% pellet | ~100% | 12 | 25 |

The references below and all the reference cited herein are incorporated herein by reference in their entireties.

### REFERENCES

[1] Moser B, Willimann K. Chemokines role in inflammation and immune surveillance. Ann Rheum Dis 2004; 63 Suppl 2: ii84-ii89.
[2] Rossi D, Zlotnik A. The biology of chemokines and their receptors Annu Rev Immunol 2000; 18: 217-42.
[3] Gerard C, Rollins B J. Chemokines and disease. Nat Immunol 2001; 2: 108-15.
[4] Lusso P. HIV and the chemokine system: 10 years later. EMBO J. 2006; 25: 447-56.
[5] O'Hayre M, Salanga C L, Handel T M, Allen S J. Chemokines and cancer: migration, intracellular signalling and intercellular communication in the microenvironment. Biochem J 2008; 409: 635-49.
[6] Park S H, Prytulla S, De Angelis A A, Brown J M, Kiefer H, Opella S J. High-resolution NMR spectroscopy of a GPCR in aligned bicelles. J Am Chem Soc 2006; 128: 7402-3.
[7] Wu B, Chien E Y, Mol C D, Fenalti G, Liu W, Katritch V, Abagyan R, Brooun A, Wells P, Bi F C, Hamel D J, Kuhn P, Handel T M, Cherezov V, Stevens R C. Structures of the CXCR4 chemokine GPCR with small-molecule and cyclic peptide antagonists. Science 2010; 330: 1066-71.
[8] Allen S J, Ribeiro S, Horuk R, Handel T M. Expression, purification and in vitro functional reconstitution of the chemokine receptor CCR1. Protein Expr Purif 2009; 66: 73-81.
[9] Blackburn P E, Simpson C V, Nibbs R J, O'Hara M, Booth R, Poulos J, Isaacs N R, Graham G J. Purification and biochemical characterization of the D6 chemokine receptor. Biochem J 2004; 379: 263-72.
[10] Mirzabekov T, Bannert N, Farzan M, Hofmann W, Kolchinsky P, Wu L, Wyatt R, Sodroski J. Enhanced expression, native purification, and characterization of CCR5, a principal HIV-1 coreceptor. J Biol Chem 1999; 274: 28745-50.
[11] Nisius L, Rogowski M, Vangelista L, Grzesiek S. Large-scale expression and purification of the major HIV-1 coreceptor CCR5 and characterization of its interaction with RANTES. Protein Expr Purif 2008; 61: 155-62.
[12] Inglese J, Samama P, Patel S, Burbaum J, Stroke I L, Appell K C. Chemokine receptor-ligand interactions measured using time-resolved fluorescence. Biochemistry 1998; 37: 2372-7.
[13] Datta-Mannan A, Stone M J. Chemokine-binding specificity of soluble chemokine-receptor analogues: identification of interacting elements by chimera complementation. Biochemistry 2004; 43: 14602-11.
[14] Zoffmann S, Turcatti G, Galzi J, Dahl M, Chollet A. Synthesis and characterization of fluorescent and photoactivatable MIP-1 alpha ligands and interactions with chemokine receptors CCR1 and CCR5. J Med Chem 2001; 44: 215-22.
[15] Strong A E, Thierry A C, Cousin P, Moulon C, Demotz S. Synthetic chemokines directly labeled with a fluorescent dye as tools for studying chemokine and chemokine receptor interactions. Eur Cytokine Netw 2006; 17: 49-59.
[16] de Paz J L, Seeberger P H. Deciphering the glycosaminoglycan code with the help of microarrays. Mol Biosyst 2008; 4: 707-11.
[17] Detheux M, Standker L, Vakili J, Munch J, Forssmann U, Adermann K, Pohlmann S, Vassart G, Kirchhoff F, Parmentier M, Forssmann W G. Natural proteolytic processing of hemofiltrate CC chemokine 1 generates a potent CC chemokine receptor (CCR)1 and CCR5 agonist with anti-HIV properties. J Exp Med 2000; 192: 1501-8.
[18] Lazar G A, Desjarlais J R, Handel T M. De novo design of the hydrophobic core of ubiquitin. Protein Sci 1997; 6: 1167-78.
[19] Catanzariti A M, Soboleva T A, Jans D A, Board P G, Baker R T. An efficient system for high-level expression and easy purification of authentic recombinant proteins. Protein Sci 2004; 13: 1331-9.
[20] Zabel B A, Allen S J, Kulig P, Allen J A, Cichy J, Handel T M, Butcher E C. Chemerin activation by serine proteases of the coagulation, fibrinolytic, and inflammatory cascades. J Biol Chem 2005; 280: 34661-6.
[21] O'Callaghan C A, Byford M F, Wyer J R, Willcox B E, Jakobsen B K, McMichael A J, Bell J I. BirA enzyme: production and application in the study of membrane receptor-ligand interactions by site-specific biotinylation. Anal Biochem 1999; 266: 9-15.
[22] Hesselgesser J, Ng H P, Liang M, Zheng W, May K, Bauman J G, Monahan S, Islam I, Wei G P, Ghannam A, Taub D D, Rosser M, Snider R M, Morrissey M M, Perez H D, Horuk R. Identification and characterization of small molecule functional antagonists of the CCR1 chemokine receptor. J Biol Chem 1998; 273: 15687-92.
[23] Vaidehi N, Schlyer S, Trabamino R J, Floriano W B, Abrol R, Sharma S, Kochanny M, Koovakat S, Dunning L, Liang M, Fox J M, de Mendonca F L, Pease J E, Goddard W A, 3rd, Horuk R. Predictions of CCR1 chemokine receptor structure and BX 471 antagonist binding followed by experimental validation. J Biol Chem 2006; 281: 27613-20.
[24] Wu S C, Wong S L. Engineering soluble monomeric streptavidin with reversible biotin binding capability. J Biol Chem 2005; 280: 23225-31.
[25] Chiu M L, Tsang C, Grihalde N, MacWilliams M P. Over-expression, solubilization, and purification of G protein-coupled receptors for structural biology. Comb Chem High Throughput Screen 2008; 11: 439-62.
[26] Hanson M A, Stevens R C. Discovery of new GPCR biology: one receptor structure at a time. Structure 2009; 17: 8-14.

The disclosure of U.S. Provisional Application No. 61/114,412 is incorporated herein by reference in its entirety. The above description discloses several methods and systems of the present invention. This invention is susceptible to modifications in the methods and materials, as well as alterations in the fabrication methods and equipment. Such modifications will become apparent to those skilled in the art from a consideration of this disclosure or practice of the invention disclosed herein. For example, the invention has been exemplified using nucleic acids but can be applied to other polymers as well. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

1. A method for producing a recombinant protein comprising:
producing a fusion protein in a cell, wherein said fusion protein comprises a protein of interest and a modified protein moiety, wherein said modified protein moiety decreases the solubility of said fusion protein in said cell compared to the solubility of said protein of interest in said cell.

2. The method of claim 1, further comprising cleaving said protein of interest from said modified protein moiety.

3. The method of claim 2, wherein said cleaving comprises utilizing a protease.

4. The method of claim 3, wherein said protease is ubiquitinase.

5. The method of claim 4, wherein said ubiquitinase comprises Usp2-cc.

6. The method of claim 1, further comprising providing conditions for said protein of interest to refold into an active form, wherein said protein of interest is cleaved from said modified protein moiety.

7. The method of claim 1, further comprising purifying said fusion protein or said protein of interest.

8. The method of claim 1, wherein said modified protein moiety comprises a modified ubiquitin moiety.

9. The method of claim 8, wherein said modified ubiquitin moiety comprises an increased frequency or number of hydrophobic amino acid residues compared to a wild type ubiquitin moiety.

10. The method of claim 8, wherein said modified ubiquitin moiety comprises a decreased frequency or number of hydrophobic amino acid residues compared to a wild type ubiquitin moiety.

11. The method of claim 8, wherein said modified ubiquitin moiety comprises a ubiquitin sequence with one or more mutations at positions selected from the group consisting of I3, V5, I13, L15, V17, I23, V26, I30, L43, L50, L56, and L69.

12. (canceled)

13. (canceled)

14. (canceled)

15. (canceled)

16. The method of claim 8, wherein said modified ubiquitin moiety comprises a sequence selected from the groups consisting of SEQ ID NO:10, SEQ ID NO:11, and SEQ ID NO:26.

17. The method of claim 1, wherein said protein of interest is a soluble protein.

18. (canceled)

19. The method of claim 1, wherein said protein of interest is a chemokine or a cytokine.

20. The method of claim 19, wherein the protein of interest comprises a polypeptide selected from the group consisting of SEQ ID NO.s:27-69.

21. The method of claim 1, wherein said protein of interest is toxic to said cell, wherein said protein of interest is in a soluble form.

22. The method of claim 1, wherein said protein of interest is degraded by said cell, wherein said protein of interest is in a soluble form.

23. The method of claim 1, wherein said protein of interest is encoded by a nucleic acid sequence codon-optimized for expression in said cell.

24. (canceled)

25. A recombinant protein produced by the method of claim 1.

26. A fusion protein comprising a protein of interest and a modified protein moiety, wherein said modified protein moiety decreases the solubility of said fusion protein in a cell compared to the solubility of said protein of interest in said cell.

27. The fusion protein of claim 26, wherein said modified protein moiety comprises a modified ubiquitin moiety.

28. The fusion protein of claim 27, wherein said modified ubiquitin moiety comprises an increased frequency of hydrophobic amino acid residues compared to a wild type ubiquitin moiety.

29. The fusion protein of claim 27, wherein said modified ubiquitin moiety comprises a decreased frequency or number of hydrophobic amino acid residues compared to a wild type ubiquitin moiety.

30. The method of claim 27, wherein said modified ubiquitin moiety comprises a ubiquitin sequence with one or more mutations at positions selected from the group consisting of I3, V5, I13, L15, V17, I23, V26, I30, L43, L50, L56, and L69.

31. (canceled)

32. (canceled)

33. (canceled)

34. (canceled)

35. The fusion protein of claim 27, wherein said modified ubiquitin moiety comprises a sequence selected from the groups consisting of SEQ ID NO:10, SEQ ID NO:11, and SEQ ID NO:26.

36. The fusion protein of claim 26, wherein said protein of interest is a soluble protein.

37. (canceled)

38. The fusion protein of claim 26, wherein said protein of interest is a chemokine or a cytokine.

39. The fusion protein of claim 38, wherein the protein of interest comprises a polypeptide selected from the group consisting of SEQ ID NO.s: 27-69.

40. The fusion protein of claim 26, wherein said protein of interest is toxic to a cell, wherein said cell expresses said fusion protein.

41. The fusion protein of claim 26, wherein said protein of interest is degraded by a cell, wherein said cell expresses said fusion protein.

42. The fusion protein of claim 26, wherein said protein of interest is encoded by a nucleic acid sequence codon-optimized for expression in said cell.

43. (canceled)

44. A nucleic acid encoding the fusion protein of claim 26.

45. A cell comprising the nucleic acid of claim 44.

46-66. (canceled)
